(19) United States
(12) Patent Application Publication
(10) Pub. No.: US 2005/0013812 A1
(43) Pub. Date: Jan. 20, 2005

(54) Vaccines using pattern recognition receptor-ligand:lipid complexes

(76) Inventors: Steven W. Dow, Littleton, CO (US); Jeffery Fairman, Mountain View, CA (US)

Correspondence Address: HOGAN & HARTSON LLP, ONE TABOR CENTER, SUITE 1500, 1200 SEVENTEENTH ST, DENVER, CO, 80202, US

(21) Appl. No.: 10/621,254

(22) Filed: Jul. 14, 2003

(51) Int. Cl. A61K 39/395

(52) U.S. Cl. 424/144.1

(57) ABSTRACT

This invention relates to a vaccine and a method for immune activation which is effective for eliciting both a systemic, non-antigen specific immune response and a strong antigen-specific immune response in a mammal. The method is particularly effective for protecting a mammal from a disease including cancer, a disease associated with allergic inflammation, an infectious disease, or a condition associated with a deleterious activity of a self-antigen. Also disclosed are therapeutic compositions useful in such a method.

### CONTRACTUAL ORIGIN OF THE INVENTION

This invention was supported in part by NIH Grant No. CA 86224-02, awarded by the National Institutes of Health. The government has certain rights to this invention.

### BACKGROUND OF THE INVENTION

1. Field of the Invention

The present invention includes compositions and methods for eliciting systemic, non-specific (i.e., non-antigen-specific) immune responses in a mammal as well as antigen-specific immune responses, both of which are useful in immunization protocols, and for eliciting angiogenesis and fibrosis formation. More particularly, the present invention relates to compositions and methods for eliciting an immune response in a mammal using liposome-toll-like receptor ligand complexes.

2. Description of the State of Art

Along with water sanitation, prevention of infectious diseases by vaccination is the most efficient, cost-effective, and practical method of disease prevention. No other modality, not even antibiotics, has had such a major effect on mortality reduction and population growth. The impact of vaccination on the health of the world's people is hard to exaggerate. Vaccination, at least in parts of the world, has controlled the following nine major diseases: smallpox, diphtheria, tetanus, yellow fever, pertussis, poliomyelitis, measles, mumps and rubella. The effectiveness of a vaccine depends upon its ability to elicit a protective immune response, which will be generally described below.

The immune response is an exceedingly complex and valuable homeostatic mechanism that has the ability to recognize foreign pathogens. The initial response to foreign pathogen is called “innate immunity” and is characterized by the rapid migration of natural killer cells, macrophages, neutrophils, and other leukocytes to the site of the foreign pathogen. These cells can either phagocytose, digest, lyse, or secrete cytokines that lyse the pathogen in a short period of time. The innate immune response is not antigen-specific and is generally regarded as a first line of defense against foreign pathogens until the “adaptive immune response” can be generated. Both T cells and B cells participate in the adaptive immune response. A variety of mechanisms are involved in generating the adaptive immune response. A discussion of all the possible mechanisms of generating the adaptive immune response is beyond the scope of this section; however, some mechanisms which have been well-characterized include B cell recognition of antigen and subsequent activation to secrete antigen-specific antibodies and T cell activation by binding to antigen presenting cells. B cell recognition involves the binding of antigen, such as bacterial cell wall, bacterial toxin, or a glyco-protein found on a viral membrane to the surface immunoglobulin receptors on B cells. The receptor binding transmits a signal to the interior of the B cell. This is what is commonly referred in the art as “first signal.” In some cases, only one signal is needed to activate the B cells. These antigens that can activate B cells without having to rely on T cell help are commonly referred to as T-independent antigens (or thymus-independent antigens). In other cases, a “second signal” is required and this is usually provided by T helper cells binding to the B cell. When T cell help is required for the activation of the B cell to a particular antigen, the antigen is then referred to as T-dependent antigen (or thymus-dependent antigen). In addition to binding to the surface receptors on the B cells, the antigen can also be internalized by the B cell and then digested into smaller fragment within the B cell and presented on the surface of B cells in the context of antigenic peptide-MHC class II molecules. These peptide-MHC class II molecules are recognized by T helper cells that bind to the B cell to provide the “second signal” needed for some antigens. Once the B cell has been activated, the B Cells begin to secrete antibodies to the antigen that will eventually lead to the inactivation of the antigen. Another way for B cells to be activated is by contact with follicular dendritic cells (FDCs) within germinal centers of lymph nodes and spleen. The follicular dendritic cells trap antigen-antibody (Ag-Ab) complexes that circulate through the lymph node and spleen and the FDCs present these to B cells to activate them.

Another well-characterized mechanism of adaptive immune response to antigens is the activation of T cells by binding to antigen presenting cells such as macrophages and dendritic cells. Macrophages and dendritic cells are potent antigen presenting cells. Macrophages have a variety of receptors that recognize microbial constituents such as macrophage mannose receptor and the scavenger receptor. These receptors bind microorganisms and the macrophage engulfs them and degrades the microorganisms in the endosomes and lysosomes. Some microorganisms are destroyed directly this way. Other microorganisms are digested into small peptides that are then presented to T cells on the surface of the macrophages in the context of MHC class II-peptide complexes. T cells that bind to these complexes become activated. Dendritic cells are also potent antigen presenting cells and present peptide-MHC class I molecules and peptide-MHC class II molecules to activate T cells.

When a B cell binds a novel antigen, the B cell is induced to undergo a developmental pathway called “isotype switching”. During the developmental changes, the plasma cells switch from producing general IgM type antibodies to producing highly specific IgG type antibodies. Within this population of cells, some undergo repeated divisions in a process called “clonal expansion”. These cells mature to become antibody factories that release immunoglobulins into the blood. When they are fully mature, they become identified as plasma cells, cells that are capable of releasing about 2,000 identical antibody molecules per second until they die, generally within 2 or 3 days after reaching maturity. Other cells within this group of clones never produce antibodies but function as memory cells that will recognize and bind that particular antigen upon encountering the antigen.

As a consequence of the initial challenge by an antigen there are now many more cells identical to the original B cell or parent cell, each of which is able to respond in the same way to the antigen as the original B cell. Consequently, if the antigen appears a second time, it will encounter one of the correct B cells sooner, and since these B cells are programmed for the specific IgG antibody, the immune response will begin sooner, accelerate faster, be more specific and produce greater numbers of antibodies. This event is considered a secondary or anamnestic response. Immunity can persist for years because memory cells survive for months or years and also because the foreign material is sometimes reintroduced in minute doses that are sufficient to constantly trigger low-level immune responses. In this way the memory cells are periodically replenished.

Following the first exposure to an antigen the response is often slow to yield antibody and the amount of antibody produced is small, i.e., the primary response. On secondary challenge with the same antigen, the response, i.e., the secondary response, is more rapid and of greater magnitude thereby achieving an immune state equal to the accelerated secondary response following re-infection with a pathogenic microorganism, which is the goal that is sought to be induced by vaccines.

Classically, active vaccines have been divided into two general classes: subunit vaccines and whole organism vaccines. Subunit vaccines are prepared from components of the whole organism and are usually developed in order to avoid the use of live organisms that may cause disease or to avoid the toxic components present in whole organism vaccines. The use of purified capsular polysaccharide material of H. influenza type b as a vaccine against the meningitis caused by this organism in humans is an example of a vaccine based upon an antigenic component. Whole organism vaccines, on the other hand, make use of the entire organism for vaccination. The organism may be killed or alive (usually attenuated) depending upon the requirements to elicit protective immunity. The pertussis vaccine, for example, is a killed whole cell vaccine prepared by treatment of Bordetella pertussis cells with formaldehyde. The use of killed cells, however, is usually accompanied by an attendant loss of immunogenic potential, since the process of killing usually destroys or alters many of the surface antigenic determinants necessary for induction of specific antibodies in the host.

In marked contrast to killed vaccines live attenuated vaccines are comprised of living organisms that are benign but typically can replicate in a host tissues and presumably express many natural target immunogens that are processed and presented to the immune system similar to a natural infection. This interaction elicits a protective response as if the immunized individual had been previously exposed to the disease. Ideally, these attenuated microorganisms maintain the full integrity of cell-surface constituents necessary for specific antibody induction yet are unable to cause disease, because, for example, they fail to produce virulence factors, grow too slowly, or do not grow at all in the host. Additionally, these attenuated strains should have substantially no probability of reverting to a virulent wild-type strain.

Classic vaccine theory implies that prophylactic inoculation with a non-lethal or attenuated pathogen will evoke an immune response capable of providing protection against infection with the same or similar pathogens on subsequent encounter. Such an approach is feasible with viruses; and to a lesser extent with bacteria, which possess a defined number of antigens. However, this is not the case with tumour cells, which may express a limitless number of antigens. In addition, unlike classical vaccine strategies, anticancer vaccines must induce an immune response after antigen exposure rather than before it. If anticancer vaccines are to be successful they must induce an immune response capable of eradicating existing disease, which will require a greater understanding of the nature of tumour antigens and of host-tumour interactions. Current vaccine concepts, such as a genetic vaccine, have been directed toward the induction of cellular immunity.

Genetic vaccines contain a DNA sequence that encodes an antigen(s) against which the immune response is to be generated. For genetic vaccines to generate an antigen-specific immune response, the gene of interest must be expressed in the mammalian host. Gene expression has been accomplished by use of viral vectors (e.g., adenovirus, poxvirus) that express the foreign gene of interest in the vaccinated patient and induce an immune response against the encoded protein. Alternatively, plasmid DNA encoding a foreign gene has been used to induce an immune response. The primary routes of administration of these so-called “naked” DNA vaccines are intramuscular or percutaneous. It is generally accepted that viral vector systems induce better immune responses than naked DNA systems, probably because the viral delivery systems induce more inflammation and immune activation than naked DNA vaccines. The propensity of viral vaccines to induce non-specific immune responses, primarily as a result of viral component recognition by the complement cascade and by the elicitation of antigen-specific immune responses against specific components of the viral vector, also represents a potential drawback, however, since such immune responses often prevent readministration of the vaccine.

Although there is considerable evidence from scientific and clinical studies that the immune system is capable of destroying cancerous tissue, in most cases the immune system either fails to recognize the tumor or the response that is generated is too weak to be effective. See, Farzaneh, et al., Immunol. Today, 19:294 (1998). While early detection may cure tumors in many cases, once the disease becomes metastatic to distant organs, it is almost always fatal. Furthermore, the disappointing results observed with chemotherapy, radiotherapy and surgery, individually or in combination, has shifted the attention of many investigators to immunological or biological agents. See, Ockert, et al., Immunol. Today, 20:63 (1999). As such, increasing the capacity of the immune system to mediate tumor regression has been a major goal in tumor immunology. Progress towards this goal has recently been aided by the identification of immunogenic tumor antigens and by a better understanding of the mechanisms of T cell-mediated immune response and tumor escape. See, Boon, et al., Immunol. Today, 18:267 (1998); Chen, Immunol. Today, 19:27 (1998).

An understanding of the mechanisms by which some animals reject tumors whereas others display progressive tumor outgrowth is gradually evolving based on an appreciation of the underlying concepts of cellular and tumor immunology. Although many tumor cells express target antigens, they are generally incapable of stimulating an immune response. See, Boon, et al., (1997); Boon, et al., J. Exp. Med., 183:725 (1996). Cytotoxic T-lymphocytes (CTL) have been recognized as a critical component of the immune response to tumors, See, Boon, et al., (1996); Chen, et al., J. Exp. Med., 179:523 (1994). CTL responses are sufficient to protect against tumors and can eliminate even established cancers in murine models (Mogi, et al., Clin. Cancer Res., 4:713 (1998)) and in humans, see, Gong, et al., Proc. Natl. Acad. Sci. USA, 97:2715 (2000). Inducing strong antigen-specific CTL responses is the goal of many current cancer vaccine strategies.

The development of CTL-dependent anti-tumor immunization strategies depends on both the identification of tumor antigens recognized by CTLs and the development of methods for effective antigen delivery. CTLs target tumors through recognition of a ligand consisting of a self MHC class I molecule and a peptide antigen generally derived from proteins synthesized within the tumor cell. However, for CTL induction and expansion to occur, the antigenic ligand must be presented to CTLs in the appropriate context of co-stimulation usually provided by professional APCs. Delivery of exogenous antigen to the endogenous MHC class I restricted processing pathway of professional APCs is a critical challenge in cancer vaccine design. Antigen delivery strategies currently under development include immunization with defined peptides, particulate proteins capable of accessing the class I pathway of professional APCs in vivo, heat shock proteins isolated from tumor cells, or adoptive transfer of antigen-loaded APCs. In addition, recent studies suggest that DNA vaccines encoding tumor antigens delivered by viral vectors or liposomes, or as naked DNA, can induce potent anti-tumor immunity.

As discussed above, methods requiring administration of peptides or proteins have inherent limitations, due to turn-over and degradation. Furthermore, generation of CTLs from CTL precursors (CTL-Ps) appears to require the interaction of IL-2 with high-affinity IL-2 receptor, resulting in proliferation and differentiation of the antigen-activated CTL-P into an effector CTL. Inadequacy of IL-2 induces Th1 cells and CTLs to undergo programmed cell death by apoptosis. In this way, the immune response is rapidly terminated, lessening the likelihood of nonspecific tissue damage from the inflammatory response.

In order to overcome the limitations of current vaccine technologies, including the CTL approach for cancers, there is an urgent need for the development of new and improved vaccine delivery systems. A likely ideal component of new and improved vaccines will be more potent vaccine adjuvants. The adjuvants to be used in these vaccines may have to closely mimic an infection and/or induce localized tissue damage to elicit protective immunity. However, current knowledge of vaccine adjuvants and how they function is still incomplete. Toll-like receptors (TLR) are thought to play a critical role in the linkage between the innate and adaptive immune systems and the development of T cell and antibody responses. However, it is still unclear exactly how activation via a specific TLR affects the type of adaptive immune response that develops. For example, activation of different TLRs may actually lead to distinct types of T cell or B cell responses.

Pattern recognition receptors, which include the Toll-like receptors, are a newly discovered family of receptors expressed by cells of the innate immune system, including macrophages, dendritic cells, and NK cells. These receptors recognize specific structural patterns on the surface of their ligands, hence the name pattern receptors. The major role for TLRs is the recognition of pathogenic microorganisms or their products and signaling to the cell following ligand binding. Signalling via TLRs causes cell activation and triggers antimicrobial defenses, including production of cytokines such as interferons, TNF, IL-12, and IL-1 and IL-6. These receptors thus serve as the body's major first defense against infectious agents. There are 12 currently identified members of this receptor family, which are also known as pattern recognition receptors. These receptors share certain common characteristics, including: (1) expression restricted primarily to antigen presenting cells of the innate immune system; (2) binding of ligands to these receptors triggers activation of immune responses against infectious pathogens (viruses, bacteria, fungi, etc.); and (3) structural similarity to the Drosophila Toll receptor. Thus, activation of cellular defenses via triggering of TLR signalling by binding of TLR ligands could serve as an effective means of inducing immune stimulation.

However, the use of TLR ligands alone for immunization may not be optimal For example, for vaccination against an antigen, simple mixing of the TLR ligand and the antigen may be a relatively inefficient means of eliciting immune responses. Moreover, administration of purified TLR ligands may result in rapid degradation in the bloodstream and may be very expensive, particularly in larger animals and humans.

There remains an urgent need to provide better vaccines which can elicit systemic, non-specific as well as antigen-specific immune responses that are safe, can be repeatedly administered, and which are effective to prevent and/or treat diseases amenable to treatment by elicitation of an immune response, such as infectious disease, allergy and cancer.

### SUMMARY OF THE INVENTION

One embodiment of the present invention relates to a vaccine. The vaccine includes the following components: (a) at least one ligand that is recognized by a pattern recognition molecule (receptor); and (b) a delivery vehicle. The ligand is complexed to or within the delivery vehicle.

Preferably, the ligand will be recognized and bound by a pattern recognition receptor molecule of the innate immunity system that elicits a cellular or humoral immune response in a mammal. The ligand may be selected for recognition by Toll-like receptors. The Toll-like receptors can include, but are not limited to, TLR-1, TLR-2, TLR-3, TLR-4, TLR-5, TLR-6, TLR-7, TLR-8, TLR-9, TLR-10, TLR-11, and TLR-12 or combinations thereof. Examples of TLR ligands can include, but are not limited to, gram⁺ bacteria (TLR-2), bacterial endotoxin TLR-4), flagellin protein (TLR-5), bacterial DNA (TLR-9), double-stranded RNA and poly (TLR-3), and yeast cell wall antigens (TLR-2). The TLR ligands used to prepare the liposome-TLR ligand complexes (LTLC) could consist of intact organisms that bind to the TLR (e.g., a gram⁺ bacterium or yeast organisms), of partially purified mixtures of proteins or carbohydrates that comprised the TLR ligands, of purified proteins or carbohydrates or lipids that comprised the TLR ligands, or of peptides or other small molecules that were capable of binding to and activating TLRs in the same manner as the native ligand. The ligands amy be more specifically glycoproteins, lipoproteins, glycolipids, carbohydrates, lipids, and/or protein or peptide sequences derived from any portion of a fungal, viral, rickettsial, parasitic, arthropod or bacterial organism. In one embodiment, the vaccine comprises multiple ligands.

One embodiment of the present invention relates to a vaccine. The vaccine includes the following components: (a) at least one immunogen for vaccinating a mammal; (b) at least one ligand that is recognized by a pattern recognition molecule (receptor); and (c) a delivery vehicle. The immunogen and the ligand are complexed to or within the delivery vehicle.

The delivery vehicle can be any suitable liposome, including, but not limited to, multilamellar vesicles, cationic liposomes, cholesterol complexed with the cationic lipids, and particularly, but not limited to, DOTMA (N-[1-(2,3-dioleoxyloxy)propyl]-N,N,N-trimethyl ammonium chloride) and cholesterol; DOTAP (1,2-Dioleoyl-3-Trimethylammonium-Propane) and cholesterol; DOTIM (1-(2-(oleoyloxy)ethyl)-2-oleyl-3-(2-hydroxyethyl)imidazolinium) and cholesterol; and DDAB (Dimethyldioctadecylammonium); PEI (Polyethylenimine), polyamines, chitosan, polyglutamic acid, protamine sulfate, microspheres, and cholesterol. In one aspect, the TLR ligands would be mixed with charged liposomes to form complexes, which would assemble spontaneously primarily due to charge-charge interactions. In most cases, the delivery vehicle to ligand molar ratio of the complexes would be greater than one, typically in the range of 8:1 to 16:1.

In one aspect, the vaccine includes a pharmaceutically acceptable excipient. Preferably the excipient includes, but is not limited to, 5-10% sucrose.

Yet another embodiment of the present invention relates to a method to elicit a systemic, non-immunogen-specific immune response in a mammal. The method includes the step of administering to the mammal a vaccine comprising: (a) at least one ligand that is recognized by a pattern recognition molecule (receptor); and (b) a delivery vehicle. The ligand is complexed to or within the delivery vehicle. The step of administering can be by any route, including, but not limited to, intravenous, intraperitoneal, subcutaneous, intradermal, intranodal, intramuscular, transdermal, inhaled, intranasal, rectal, vaginal, urethral, topical, oral, intraocular, intraarticular, intracranial, and intraspinal. In one embodiment, the step of administering is by a combination of intravenous and intranodal administration. In another aspect, the step of administering is by a combination of intraperitoneal and intranodal administration. In yet another aspect, the step of administering is by a combination of intradermal and intranodal administration.

In one aspect, the composition of the present invention is administered at a dose of from about 1 μg per individual mammal to about 1 mg per individual mammal. In another aspect, the composition of the present invention is administered at a dose of from about 1 μg per individual mammal to about 100 μg per individual mammal. In yet another aspect, the composition of the present invention is administered at a dose of from about 1 μg per individual mammal to about 10 μg per individual mammal. Preferably, administration of the vaccine to the mammal produces a result selected from the group consisting of immunization against the disease or condition and stimulation of effector cell immunity against the disease or condition.

### BRIEF DESCRIPTION OF THE DRAWINGS

The accompanying drawings, which are incorporated in and form a part of the specification, illustrate the preferred embodiment of the present invention, and together with the description serve to explain the principles of the invention.

In the Drawings:

FIG. 1 graphically illustrates that liposomes markedly enhance activation of innate immunity and INF-γ release after activation by pattern recognition receptor ligands (PRRL).

FIG. 2 Liposomes alter release of IL-10 after activation by pattern recognition receptor ligands (PRRL).

FIG. 3 graphically illustrates that liposomes enhance release of TNF-α after activation by pattern recognition receptor ligands (PRRL).

FIG. 4 graphically illustrates that liposomes alter the regulation of dendritic cell activation following exposure to pattern recognition receptor ligands (PRRL) in vitro.

FIG. 5 illustrates peptides or protein antigens complexed to lipid-DNA complexes.

FIG. 6 illustrates LANAC and “cross-priming”.

FIGS. 7A and 7B graphically illustrate the efficacy of LANAC vaccines in eliciting CTL responses compared to other conventional vaccines.

FIG. 8 graphically illustrates that liposomes enhance the ability of PRRL to serve as vaccine adjuvants for elicitation of CTL responses.

FIG. 9 graphically illustrates that liposome-PRRL complexes also act as effective vaccine adjuvants for eliciting CTL responses in the lungs.

FIG. 10 illustrates the determination of whether 3-part liposome-antigen-nucleic acid complex is required for efficient immunization.

FIGS. 11A and 11B graphically illustrate the functional capabilities of T cells elicited by immunization with liposome-nucleic acid complexes.

FIG. 12 graphically illustrates the ability of liposome-nucleic acid vaccination to elicit humoral immunity.

FIG. 13 graphically illustrates assessing the T cell memory response to vaccination with LANAC.

FIG. 14 illustrates the evaluation of the ability of mucosally-administered LANAC to elicit local and systemic immunity.

FIG. 15 illustrates assessing and comparing the efficiency of distribution of LANAC to lymphoid organs.

### DETAILED DESCRIPTION OF INVENTION

The present invention generally relates to a novel immunization strategy and therapeutic compositions for eliciting an immune response in a mammal, and in particular, in a mammal that has a disease amenable to treatment by elicitation of an immune response. Diseases which are particularly amenable to treatment using the method of the present invention include, autoimmune diseases, cancer, allergic inflammation and infectious disease. In one embodiment, the method and composition of the present invention are particularly useful for the prevention and treatment of primary lung cancers, pulmonary metastatic diseases, allergic asthma and viral diseases. In another embodiment, the method and composition of the present invention are useful for modulating angiogenesis and fibrosis formation that is useful in wound healing and for treating cardiovascular disorders, and bone disorders. The method and compositions of the present invention are further useful for modulating the immune response in a subject disposed of an autoimmune disease. In addition, elicitation of an immune response according to the method of the present invention can be useful for the development and implementation of immunological diagnostic and research tools and assays.

More particularly, the genetic immunization method of the present invention comprises the elicitation of an immune response in a mammal by intravenous or intraperitoneal administration (i.e., systemic administration) of a therapeutic composition which includes at least one ligand capable of being bound by a pattern recognition molecule (pattern recognition receptor ligand (PRRL) complexed with a delivery vehicle. Eliciting or modulating an immune response comprises augmenting an immune response or down regulating (suppressing) an immune response.

Pattern recognition receptors, which include the Toll-like receptors, (TLRs) are a newly discovered family of receptors expressed by cells of the inate immunity system, including macrophages, dentritic cells and NK cells. Examples of known ligands for TLRs include gram⁺ bacteria (TLR-2), bacterial endotoxin TLR-4), flagellin protein (TLR-5), bacterial DNA (TLR-9), double-stranded RNA and poly I:C (TLR-3), and yeast (TLR-2). Other ligands that bind an endocytic pattern recognition receptors, a scavenger receptor or a mannose-binding receptor may also be used. Accordingly, the present invention may utilize any pattern recognition receptor ligand, however, by way of example, the present invention will be described in relation to TLR ligands.

The TLR ligands used to prepare the liposome-TLR ligand complexes (LTLC) could consist of intact organisms that bind to the TLR (e.g., a gram⁺ bacterium or yeast organisms), of partially purified mixtures of proteins or carbohydrates that comprised the TLR ligands, of purified proteins or carbohydrates or lipids that comprised the TLR ligands, or of peptides or other small molecules that were capable of binding to and activating TLRs in the same manner as the native ligand. The PRRLs would be mixed with charged liposomes to form complexes, which would assemble spontaneously primarily due to charge-charge interactions. In most cases, the liposome to PRRL molar ratio of the complexes would be greater than one, typically in the range of 8:1 to 16:1.

The therapeutic composition of the present invention also includes a delivery vehicle. According to the present invention, the delivery vehicle comprises a lipid composition that is capable of fusing with the plasma membrane of a cell, thereby allowing the liposome to deliver a pattern recognition receptor ligand (PRRP) and/or a nucleic acid into a cell. A liposome is also capable of either incorporating an immunogen on its surface or incorporating the immunogen internally. Suitable delivery vehicles for use with the present invention include any liposome. In fact, the present inventors have demonstrated that the immune stimulatory effect of the combination of liposomes and PRRL is not limited to a particular type of liposome. Some preferred liposomes of the present invention include those liposomes commonly used in, for example, gene delivery methods known to those of skill in the art. Some-preferred delivery vehicles comprise multilamellar vesicle (MLV) lipids and extruded lipids, although the invention is not limited to such liposomes.

Methods for preparation of MLV's are well known in the art and are described, for example, in U.S. patent application Ser. No. 09/104,759, ibid. According to the present invention, “extruded lipids” are lipids which are prepared similarly to MLV lipids, but which are subsequently extruded through filters of decreasing size, as described in Templeton, et al., Nature Biotech., 15:647-652 (1997), which is incorporated herein by reference in its entirety. Small unilamellar vesicle (SWV) lipids can also be used in the composition and method of the present invention and have been shown to be effective in combination with nucleic acids for eliciting an immune response (see U.S. patent application Ser. No. 09/104,759, ibid.). Other preferred liposome delivery vehicles comprise liposomes having a polycationic lipid composition (i.e., cationic liposomes). For example, cationic liposome compositions include, but are not limited to, any cationic liposome complexed with cholesterol, and without limitation, include DOTMA (N-[1-(2,3-dioleoxyloxy)propyl]-N,N,N-trimethyl ammonium chloride) and cholesterol, DOTAP (1,2-Dioleoyl-3-Trimethylammonium-Propane) and cholesterol, DOTIM (1-(2-(oleoyloxy)ethyl)-2-oleyl-3-(2-hydroxyethyl)imidazolinium) and cholesterol, PEI (Polyethylenimine) and cholesterol and DDAB (Dimethyldioctadecylammonium) and cholesterol.

Liposomes of the present invention can be any size, including from about 10 and 1000 nanometers (nm), or any size in between. The liposome component of the present invention most preferably consists of charged liposomes that are comprised of a mixture of a charged lipid mixed with a neutral lipid such as cholesterol. The net charge of the liposomes would be varied according the charge of the TLR ligand, in order to maximize charge-charge interactions between the two. For example, to prepare complexes using bacterial DNA as the TLR ligand, cationic liposomes would be used because the DNA has a net negative charge. For uncharged TLR ligands, cationic liposomes would be preferred because of their targeting to antigen presenting cells. The liposomes would also in most cases be formulated as modified multilamellar liposomes, with a size appropriate for the desired route of delivery. For the sake of clarity the term liposome will be used throughout the remainder of this section when describing the delivery vehicle copnent of the present invention. However, it is to be understood that the liposome component may be substituted with any of the delivery vehicles described above.

A liposome delivery vehicle of the present invention can be modified to target a particular site in a mammal, thereby targeting and making use of a nucleic acid molecule of the present invention at that site. Suitable modifications include manipulating the chemical formula of the lipid portion of the delivery vehicle. Manipulating the chemical formula of the lipid portion of the delivery vehicle can elicit the extracellular or intracellular targeting of the delivery vehicle. For example, a chemical can be added to the lipid formula of a liposome that alters the charge of the lipid bilayer of the liposome so that the liposome fuses with particular cells having particular charge characteristics. In one embodiment, other targeting mechanisms, such as targeting by addition of exogenous targeting molecules to a liposome (i.e., antibodies) may not be a necessary component of the liposome of the present invention, since effective immune activation at immunologically active organs can already be provided by the composition when the route of delivery is intravenous or intraperitoneal, without the aid of additional targeting mechanisms. However, in some embodiments, a liposome can be directed to a particular target cell or tissue by using a targeting agent, such as an antibody, soluble receptor or ligand, incorporated with the liposome, to target a particular cell or tissue to which the targeting molecule can bind. Targeting liposomes are described, for example, in Ho, et al., Biochemistry, 25:5500-6 (1986); Ho, et al., J Biol Chem, 262:13979-84 (1987); Ho, et al., J Biol Chem, 262:13973-8 (1987); and U.S. Pat. No. 4,957,735 to Huang, et al., each of which is incorporated herein by reference in its entirety). In one embodiment, if avoidance of the efficient uptake of injected liposomes by reticuloendothelial system cells due to opsonization of liposomes by plasma proteins or other factors is desired, hydrophilic lipids, such as gangliosides (Allen, et al., FEBS Lett, 223:42-6 (1987)) or polyethylene glycol (PEG)-derived lipids (Klibanov, et al., FEBS Lett, 268:235-7 (1990)), can be incorporated into the bilayer of a conventional lipo some to form the so-called sterically-stabilized or “stealth” liposomes (Woodle, et al., Biochim Biophys Acta, 1113:171-99 (1992)). Variations of such liposomes are described, for example, in U.S. Pat. No. 5,705,187 to Unger, et al., U.S. Pat. No. 5,820,873 to Choi, et al., U.S. Pat. No. 5,817,856 to Tirosh, et al.; U.S. Pat. No. 5,686,101 to Tagawa et al.; U.S. Pat. No. U.S. Pat. No. 5,043,164 to Huang, et al., and U.S. Pat. No. 5,013,556 to Woodle, et al., all of which are incorporated herein by reference in their entireties).

As discussed above, a vaccine or therapeutic composition of the present invention is administered to a mammal in a manner effective to deliver the composition to a cell, a tissue, and/or systemically to the mammal, whereby elicitation of an immunogen-specific immune response is achieved as a result of the administration of the composition. It is noted that while it is possible to specifically target the therapeutic composition of the present invention to a particular cell or tissue, it is not necessary, since the inventors have found that several different modes of administration in the absence of specific targeting is effective to elicit the desired immune response. Suitable administration protocols include any in vivo or ex vivo administration protocol. According to the present invention, suitable methods of administering a vaccine or therapeutic composition of the present invention to a patient include any route of in vivo administration that is suitable for delivering the composition into a patient. The preferred routes of administration will be apparent to those of skill in the art, depending on the type of condition to be prevented or treated, the immunogen used, and/or the target cell population. Preferred methods of in vivo administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracranial, intraspinal, intraocular, intranasal, oral, bronchial, rectal, topical, vaginal, urethral, pulmonary administration, impregnation of a catheter, and direct injection into a tissue. In particular, any routes of delivery which elicit an immune response in the mucosal tissues is preferred. Such routes include bronchial, intradermal, intramuscular, intranasal, other inhalatory, rectal, subcutaneous, topical, transdermal, vaginal and urethral routes. Some particularly preferred routes of administration include, intravenous, intraperitoneal, subcutaneous, intradermal, intranodal, intramuscular, transdermal, inhaled, intranasal, rectal, vaginal, urethral, topical, oral, intraocular, intraarticular, intracranial, and intraspinal. As discussed previously, combinations of routes of delivery can be used and in some instances, may enhance the therapeutic effects of the vaccine or composition. Therefore, any combination of two or more routes of administration, performed simultaneously, within a short time period one after another, or at different time intervals relative to the immunization schedule (e.g., initial administration versus boosters), are contemplated by the present inventors. In one embodiment, a preferred route of administration is a combination of any one or more of intravenous, intraperitoneal or intradermal administration with intranodal administration. In another embodiment where the target cells are in or near a tumor, a preferred route of administration is by direct injection into the tumor or tissue surrounding the tumor.

Ex vivo administration refers to performing part of the regulatory step outside of the patient, such as administering a composition of the present invention to a population of cells removed from a patient under conditions such that the composition contacts and/or enters the cell, and returning the lipofected cells to the patient. Ex vivo methods are particularly suitable when the target cell can easily be removed from and returned to the patient.

A therapeutic composition or vaccine according to the present invention may be prepared using the PRRL-liposome complexes described above, together with an antigen for the purposes of enhancing immune responses against that antigen (i.e., a vaccine). Thus, in this embodiment the PRRL:liposome complex would serve as a vaccine adjuvant. For the purposes of this invention the antigen to be immunized against could consist of intact microorganisms or cells, partially disrupted microorganisms or cells, lysates prepared from microorganisms or cells, purified proteins, carbohydrates or lipids or complex mixtures thereof derived from microorganisms or cells, or peptide antigens derived from microorganisms or cells. The term “cells” in this disclosure refers primarily to either autologous or allogeneic tumor cells, for the purposes of preparing a tumor vaccine. “Microorganisms” refers to either viral, bacterial, fungal, protozoal, or parasitic pathogens.

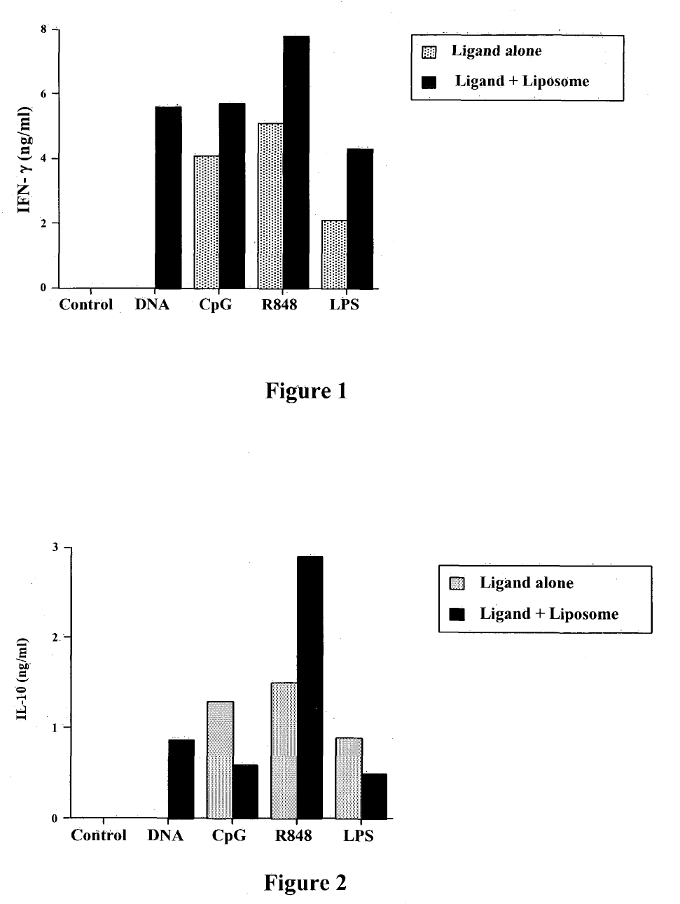

The use of PRRLs alone for immunization may not be optimal. For example, for vaccination against an antigen, simple mixing of the TLR ligand and the antigen may be a relatively inefficient means of eliciting immune responses, See FIGS. 1-4. Moreover, administration of purified TLR ligands may result in rapid degradation in the bloodstream and may be very expensive, particularly in larger animals and humans. The composition of the present invention makes use of a delivery vehicle, such as but not limited to a liposome as one very effective method to potentiate the effectiveness of TLR ligands, particularly for immune activation (both local and systemic) and for eliciting T cell responses. Liposomes administered in conjunction with TLR ligands serve two purposes. For one, the combination of a liposomes and TLR ligand can serve to greatly potentiate the inherent immunostimulatory properties of the TLR ligand via a synergistic immunostimulatory interaction with the liposomes. Secondly, for a vaccine the liposomes can serve as a physical means of bringing the TLR ligand and the antigen into close proximity. This in turn assures that the same antigen presenting cell that is activated by the TLR ligand is also presenting the antigen to T cells and B cells.

Administration of liposome-TLR ligand complexes (LTLC) for immune activation. Based on prior work using LTLC formulated with one TLR ligand (bacterial DNA), it is expected that LTLC prepared with other TLR ligands will be highly immunostimulatory. In particular, the combination of the liposomes and the TLR will be much more stimulatory than either component alone. However, it is also likely that use of different TLR ligands to stimulate different TLRs will induce immune responses that differ both quantitatively and qualitatively. Therefore, use of different LTLC formulations can be used to selectively manipulate the types of immune responses that are elicited. The LTLC can be administered by a variety of routes, depending on whether systemic or local immune stimulation is desired. For example, maximal systemic immune activation can be achieved by intravenous or intraperitoneal administration of LTLC, whereas inhalation of LTLC can be used to induce local immune activation in the lungs. Preferred routes of administration would be inhalation, intravenous, oral, and intraperitoneal.

Immune activation by LTLC can be used to treat a variety of diseases that can be ameliorated by strong activation of innate immunity. One therapeutic application of LTLC would be in the treatment of cancer, in a variety of sites including the lungs, skin, liver, peritoneal cavity, and bone marrow. A second application would be in the treatment or prevention of infectious diseases, including viral, fungal, and bacterial infections of the lungs or airways, or other sites. Another application would be in the prevention or treatment of allergic diseases, including asthma and allergic rhinitis. Another application would be to produce hematopoetic differentiation or hematopoetic remodling which would serve as or facilitate vaccine and/or immunotherapeutic treatment.

Vaccination using LTLC and antigens. The immune stimulatory properties of LTLC also make them very effective as adjuvants for boosting immune responses against antigens in vaccines. The adjuvant properties of LTLC would increase both T cell responses and antibody responses to the vaccine antigen. To prepare a vaccine using LTLC, the antigen to be immunized against can be added to preformed LTLC, or can first be incorporated with the liposomes and then mixed with the TLR ligand.

Vaccines prepared from LTLC plus antigens could be administered in a variety of routes, including conventional routes (IM, SC, ID). Moreover, the vaccines could applied to mucosal surfaces to induce local immune responses. For example, an inhaled LTLC vaccine could be used to elicit pulmonary immune responses against an antigen. Vaccines prepared using LTLC could also be repeatedly administered, without induction of harmful immunity against the LTLC component of the vaccine. The antigen(s) used in the LTLC vaccine could consist of proteins peptides, carbohydrates, lipoproteins, or complex mixtures of any or all of the foregoing. The antigens could be derived from cell lysates, pathogenic organism lysates, or any purified or synthesized component of those cells or organisms. In addition, the vaccine could also be prepared using normal cells or cell proteins to elicit therapeutic cross-reacting immune responses against normal cellular proteins. Examples of this latter application would include immunization against β-amyloid proteins for therapeutic modulation of Alzheimer's disease. The vaccine could also be used against disease caused by an abnormal production Of proteins in a specific area of the body, such as but not limited to the brain, kidneys, Or joints.

The use of delivery vehicles, such as but not limited to, liposomes is one effective method to potentiate the effectiveness of TLR ligands, particularly for immune activation (both local and systemic) and for eliciting T cell responses. Liposomes administered in conjunction with TLR ligands serve two purposes. For one, the combination of a liposomes and TLR ligand can serve to greatly potentiate the inherent immunostimulatory properties of the TLR ligand via a synergistic immunostimulatory interaction with the liposomes. Secondly, for a vaccine the liposomes can serve as a physical means of bringing the TLR ligand and the antigen into close proximity. This in turn assures that the same antigen presenting cell that is activated by the TLR ligand is also presenting the antigen to T cells and B cells.

The present inventors have made the surprising discovery that the combination of PRRL and liposomes is highly immunostimulatory in vivo when administered by intravenous or intraperitoneal injection. The potency of this immune response is far greater than the response induced by administration of either ligands or liposomes alone (See Examples 1-4), and is dependent upon the intravenous or intraperitoneal administration of the complex. As such, the PRRL-lipid complexes of the present invention induce a strong, systemic, non-antigen-specific immune response when administered intravenously or intraperitoneally, which results in the activation of multiple different immune effector cells in vivo. The present inventors have additionally discovered that the immune response generated by such a LTLC administered by the present method has potent anti-tumor, anti-allergy and anti-viral properties. Immune activation induced by such a therapeutic composition of the present invention is quantitatively more potent than that induced by either LPS (endotoxin) or poly I/C (a classical inducer of antiviral immune responses. Furthermore, the type of immune stimulation induced (e.g., as characterized by the pattern of cytokines induced) also differs qualitatively from that induced by LPS or poly I/C. Finally, this effect does not appear to be associated with the complement cascade problems that have been experienced using viral delivery systems.

At present however we know little about the immunologic mechanisms that underlie the efficacy of these LTLC adjuvants. Endocytosis of the charged liposomes most likely serves to introduce both the antigen and the TLR-ligand into the endosomal compartment of the antigen-presenting cell, where both antigen processing and TLR activation can occur. The properties of this unique adjuvant system, which elicits very potent T cell responses, will be exploited for use as a tool to study the effects of activation of different TLRs on antigen presentation and induction of adaptive immunity. Different LTLC will be evaluated first for their affects on innate immunity, then with model antigens and Yersinia proteins for elicitation of adaptive and protective immunity against Yersinia pestis. These studies will enhance our basic understanding of vaccine adjuvants and the role of TLR activation in general and will also help develop more effective mucosal vaccines against aerosolized pathogens such as Yersinia.

When the route of administration is intravenous, the primary site of immunization (i.e., elicitation of an immune response) is the lung, which is a very active organ immunologically, containing large numbers of both effector cells (e.g., T cells, B cells, NK cells) and antigen presenting cells (e.g., macrophages, dendritic cells). Similarly, when the route of administration is intraperitoneal, the primary sites of immunization are the spleen and liver, both of which are also immunologically active organs.

Due to the unexpected immunostimulatory properties of the TLR-ligand:liposome complexes administered by the present method, the genetic immunization method of the present invention is particularly useful in human treatments because traditional adjuvants can be avoided. This is a particular advantage of the present method, since some traditional adjuvants can be toxic (e.g., Freund's adjuvant and other bacterial cell wall components) and others are relatively ineffective (e.g., aluminum-based salts and calcium-based salts). Moreover, the only adjuvants currently approved for use in humans in the United States are the aluminum salts, aluminum hydroxide and aluminum phosphate, neither of which stimulates cell-mediated immunity. In addition, as will be shown in the Examples below, traditional naked DNA delivery, which has been touted as having an adjuvant effect, is far less effective than the present compositions at stimulating a non-antigen-specific immune response. Finally, unlike many protocols for administration of viral vector-based genetic vaccines, the present method can be used to repeatedly deliver the therapeutic composition described herein without consequences associated with some non-specific arms of the immune response, such as the complement cascade.

In further embodiments of the present invention, the present inventors have taken advantage of the non-antigen-specific immunostimulatory effect of the above-described method and have developed an even more powerful genetic immunization strategy in which a nucleic acid sequence encoding an immunogen and/or a cytokine that is expressed in the tissues of the mammal (i.e., is operatively linked to a transcription control sequence) is further complexed with the LTLC. The present inventors have also found that the combination of an antigen-specific immune response elicited by expression of an immunogen, in conjunction with the powerful, non-antigen specific immune response elicited by the LTLC results in a vaccine that has significantly greater in vivo efficacy than previously described genetic vaccines (See Examples 5, 6b-c, 9). This effect can be additionally enhanced by co-administration of a nucleic acid molecule encoding a cytokine such that the cytokine is expressed in the tissues.

Moreover, with regard to intravenous administration of the present composition, in cancer patients, the lung is the principal site to which metastatic tumors spread. The method of the present invention is particularly successful in mammals having cancer, because it induces a strong enough immune response to reduce or eliminate a primary tumor and to control any metastatic tumors that are already present, including large metastatic tumors. Therefore, the genetic immunization method and compositions of the present invention, unlike previously described genetic immunization methods, elicit both a systemic, non-antigen-specific immune response (similar to a conventional adjuvant) and, when the nucleic acid encodes a tumor antigen, a strong, antigen-specific, intrapulmonary (intravenous administration; see Example 9 immune response in a mammal which is effective to significantly reduce or eliminate established tumors in vivo.

One embodiment of the present invention is a method to elicit a systemic, non-antigen-specific immune response in a mammal immune response in a mammal. In this method, a therapeutic composition which includes: (a) a delivery vehicle; and (b) a pattern recognition receptor ligand (PRRL) complexed to or within the delivery vehicle, is administered by intravenous or intraperitoneal administration to a mammal. Administration of such a composition by the method of the present invention results in the elicitation of a systemic, non-antigen-specific immune response in the mammal to which the composition is administered. As discussed above, this immune response additionally has strong, systemic, anti-tumor, anti-allergic inflammation (i.e., protective), and anti-viral properties.

Therapeutic compositions useful in the method of the present invention include compositions containing PRRL, including TLR-Ligands, such as but not limited to gram+ bacterium or yeast organisms), of partially purified mixtures of proteins or carbohydrates that comprised the TLR ligands, of purified proteins or carbohydrates or lipids that comprised the TLR ligands, or of peptides or other small molecules that were capable of binding to and activating TLRs in the same manner as the native ligand. The TLR ligands would be mixed with charged liposomes to form complexes, which would assemble spontaneously primarily due to charge-charge interactions.

In another embodiment of the present invention, the present method of eliciting an immune response can be modified to include the intravenous or intraperitoneal administration to a mammal of a therapeutic composition comprising: (a) a PRRL; (b) a delivery vehicle; and (c) a recombinant nucleic acid molecule comprising a nucleic acid sequence which encodes an immunogen. According to the present invention, the terms “immunogen” and “antigen” can be used interchangeably, although the term “antigen” is primarily used herein to describe a protein which elicits a humoral and/or cellular immune response (i.e., is antigenic), and the term “immunogen” is primarily used herein to describe a protein which elicits a humoral and/or cellular immune response in vivo, such that administration of the immunogen to a mammal mounts an immunogen-specific (antigen-specific) immune response against the same or similar proteins that are encountered within the tissues of the mammal. According to the present invention, an immunogen or an antigen can be any portion of a protein, naturally occurring or synthetically derived, which elicits a humoral and/or cellular immune response. As such, the size of an antigen or immunogen can be as small as about 5-12 amino acids and as large as a full length protein, including a multimer and fusion proteins. The terms, “immunogen” and “antigen”, as used to describe the present invention, do not include a superantigen. A superantigen is defined herein as the art-recognized term. More particularly, a superantigen is a molecule within a family of proteins that binds to the extracellular portion of an MHC molecule (i.e., not in the peptide binding groove) to form and MHC:superantigen complex. The activity of a T cell can be modified when a TCR binds to an MHC:superantigen complex. Under certain circumstances, an MHC:superantigen complex can have a mitogenic role (i.e., the ability to stimulate the proliferation of T cells) or a suppressive role (i.e., deletion of T cell subsets).

In preferred embodiments, the immunogen is selected from the group of a tumor antigen, an allergen or an antigen of an infectious disease pathogen (i.e., a pathogen antigen). In this embodiment, the nucleic acid sequence is operatively linked to a transcription control sequence, such that the immunogen is expressed in a tissue of a mammal, thereby eliciting an immunogen-specific immune response in the mammal, in addition to the non-specific immune response discussed above.

In a further embodiment of the method of the present invention, the therapeutic composition to be administered to a mammal includes an isolated nucleic acid molecule encoding a cytokine (also referred to herein as a “cytokine-encoding nucleic acid molecule”), in which the nucleic acid molecule is operatively linked to one or more transcription control sequences. The result of administration of such a therapeutic composition to the mammal is that the nucleic acid molecule encoding the cytokine is expressed in the pulmonary tissues of the mammal, when administration is intravenous, and in the spleen and liver tissues of the mammal when administration is peritoneal. It is to be noted that the term “a” or “an” entity refers to one or more of that entity; for example, a cytokine refers to one or more cytokines. As such, the terms “a” (or “an”), “one or more” and “at least one” can be used interchangeably herein. The nucleic acid sequence encoding a cytokine can be on the same recombinant nucleic acid molecule as a nucleic acid sequence encoding an immunogen, or on a different recombinant nucleic acid molecule.

A composition useful in the method of the present invention, as discussed in detail below, comprises: (a) a delivery vehicle; (b) a TLR-ligand. In addition the composition may further comprise a nucleic acid molecule, such molecule including: (1) an isolated nucleic acid sequence that is not operatively linked to a transcription control sequence; (2) an isolated non-coding nucleic acid sequence; (3) an isolated recombinant nucleic acid molecule encoding an immunogen operatively linked to a transcription control sequence, wherein the liposome to TLR molar ratio of the complexes would be greater than one, typoically in the range of about 8:1 to about 15:1 nucleic acid:liposome complex has a ratio of from about 1:1 to about 1:64; and/or (4) an isolated recombinant nucleic acid molecule encoding a cytokine. Various components of such a composition are described in detail below.

Elicitation of an immune response in a mammal can be an effective treatment for a wide variety of medical disorders, and in particular, for cancer, allergic inflammation and/or infectious disease. As used herein, the term “elicit” can be used interchangeably with the terms “activate”, “stimulate”, “generate” or “upregulate”. According to the present invention, “eliciting an immune response” in a mammal refers to specifically controlling or influencing the activity of the immune response, and can include activating an immune response, upregulating an immune response, enhancing an immune response and/or altering an immune response (such as by eliciting a type of immune response which in turn changes the prevalent type of immune response in a mammal from one which is harmful or ineffective to one which is beneficial or protective. For example, elicitation of a Th1-type response in a mammal that is undergoing a Th2-type response, or vice versa, may change the overall effect of the immune response from harmful to beneficial. Eliciting an immune response which alters the overall immune response in a mammal can be particularly effective in the treatment of allergic inflammation, mycobacterial infections, or parasitic infections. According to the present invention, a disease characterized by a Th2-type immune response (alternatively referred to as a Th2 immune response), can be characterized as a disease which is associated with the predominant activation of a subset of helper T lymphocytes known in the art as Th2-type T lymphocytes (or Th2 lymphocytes), as compared to the activation of Th1-type T lymphocytes (or Th1 lymphocytes). According to the present invention, Th2-type T lymphocytes can be characterized by their production of one or more cytokines, collectively known as Th2-type cytokines. As used herein, Th2-type cytokines include interleukin-4 (IL-4), interleukin-5 (IL-5), interleukin-6 (IL-6), interleukin-9 (IL-9), interleukin-10 (IL-10), interleukin-13 (IL-13) and interleukin-15 (IL-15). In contrast, Th1-type lymphocytes produce cytokines which include IL-2 and IFN. Alternatively, a Th2-type immune response can sometimes be characterized by the predominant production of antibody isotypes which include IgG1 (the approximate human equivalent of which is IgG4) and IgE, whereas a Th1-type immune response can sometimes be characterized by the production of an IgG2a or an IgG3 antibody isotype (the approximate human equivalent of which is IgG1, IgG2 or IgG3).

Preferably, the method of the present invention elicits an immune response against a tumor, an allergen or an infectious disease pathogen. In particular, eliciting an immune response in a mammal refers to regulating cell-mediated immunity (i.e., helper T cell (Th) activity, cytotoxic T lymphocyte (CTL) activity, NK cell activity) and/or humoral immunity (i.e., B cell/immunoglobulin activity), including Th1-type and/or Th2-type cellular and/or humoral activity. In a preferred embodiment, the method of the present invention increases or elicits effector cell immunity against a tumor, an allergen or an infectious disease pathogen. As used herein, effector cell immunity refers to increasing the number and/or the activity of effector cells in the mammal to which a composition is administered. In particular, T cell activity refers to increasing the number and/or the activity of T cells in the area of the tumor cell or pathogen. Similarly, NK cell activity refers to increasing the number and/or activity of NK cells. In the method of the present invention, effector cell immunity is elicited both systemically and in the area of the mammal in which the therapeutic composition is primarily targeted (i.e., intrapulmonary for intravenous administration and in the spleen or liver for intraperitoneal administration, although the present composition is effective at other sites in the body as well). According to the present invention, an effector cell includes a helper T cell, a cytotoxic T cell, a B lymphocyte, a macrophage, a monocyte and/or a natural killer cell. For example, the method of the present invention can be performed to increase-the number of effector cells in a mammal that are capable of killing a target cell or releasing cytokines when presented with antigens derived from a tumor cell, an allergen or a pathogen.

According to the present invention, elicitation of a non-antigen-specific immune response (i.e., a non-specific immune response) includes stimulation of non-specific immune cells, such as macrophages and neutrophils, as well as induction of cytokine production, particularly IFN production, and non-antigen-specific activation of effector cells such as NK cells, B lymphocytes and/or T lymphocytes. More specifically, the systemic, non-antigen-specific immune response elicited by the method and composition of the present invention result in an increase in natural killer (NK) cell function and number in the mammal, wherein an increase in NK function is defined as any detectable increase in the level of NK cell function compared to NK cell function in mammals not immunized with a composition of the present invention, or in mammals immunized with a composition of the present invention by a non-systemic (i.e., non-intravenous, non-intraperitoneal) route of administration, with the amount of TLR-ligand delivered and the ratio of TLR-ligand; liposome being equal. NK function (i.e., activity) can be measured by cytotoxicity assays against a suitable target cell. An example of an NK cell cytotoxicity assay is presented in Example 1 (FIG. 1). NK cell activation can be measured by determining an upregulation of NK1.1/CD69 on cells in various organs, including spleen, lymph node, lung and liver, by flow cytometric analysis (See Example 4, FIG. 4). Additionally, the systemic, non-antigen-specific immune response elicited by the method and composition of the present invention can result in an increase in production of IFN-γ by the NK cells in the mammal in various organs including spleen and lung, wherein an increase in IFN production is defined as any detectable increase in the level of IFN-γ production compared to IFN-γ production by NK cells in mammals not administered with a composition of the present invention, or in mammals administered with a composition of the present invention by a non-systemic route of administration, with the amount of TLR-ligand delivered and the ratio of TLR-ligand:liposome being equal. IFN-γ production can be measured by a IFN-γ ELISA (as is known in the art; Example 1, FIG. 1). Preferably, a composition of the present invention administered by the method of the present invention elicits at least about 100 μg/ml of IFN-γ per 5×10⁶ mononuclear cells from blood, spleen or lung, and more preferably, at least about 500 μg/ml of IFN-γ, and more preferably at least about 1000 μg/ml of IFN-γ, and even more preferably, at least about 5000 μg/ml of IFN-γ, and even more preferably, at least about 10,000 μg/ml of IFN-γ.

Accordingly, the method of the present invention preferably elicits an immune response in a mammal such that the mammal is protected from a disease that is amenable to elicitation of an immune response, including cancer, allergic inflammation and/or an infectious disease and/or prevention of onset of the disease. As used herein, the phrase “protected from a disease” refers to reducing the symptoms of the disease; reducing the occurrence of the disease, and/or reducing the severity of the disease. Protecting a mammal can refer to the ability of a therapeutic composition of the present invention, when administered to a mammal, to prevent a disease from occurring and/or to cure or to alleviate disease symptoms, signs or causes. As such, to protect a mammal from a disease includes both preventing disease occurrence (prophylactic treatment) and treating a mammal that has a disease (therapeutic treatment). In particular, protecting a mammal from a disease is accomplished by eliciting an immune response in the mammal by inducing a beneficial or protective immune response which may, in some instances, additionally suppress (e.g., reduce, inhibit or block) an overactive or harmful immune response. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

More specifically, a therapeutic composition as described herein, when administered to a mammal by the method of the present invention, preferably produces a result which can include alleviation of the disease, elimination of the disease, reduction of a tumor or lesion associated with the disease, elimination of a tumor or lesion associated with the disease, prevention of a secondary disease resulting from the occurrence of a primary disease (e.g., metastatic cancer resulting from a primary cancer), prevention of the disease, and stimulation of effector cell immunity against the disease.

One component of the therapeutic composition used in the present method is a nucleic acid sequence, which can include coding and/or non-coding nucleic acid sequences, and both oligonucleotides (described below) and larger nucleic acid sequences. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably. As used herein, a “coding” nucleic acid sequence refers to a nucleic acid sequence which encodes at least a portion of a peptide or protein (e.g. a portion of an open reading frame), and can more particularly refer to a nucleic acid sequence encoding a peptide or protein which is operatively linked to a transcription control sequence, so that the peptide or protein can be expressed. A “non-coding” nucleic acid sequence refers to a nucleic acid sequence which does not encode any portion of a peptide or protein. According to the present invention, “non-coding” nucleic acids can include regulatory regions of a transcription unit, such as a promoter region. The term, “empty vector” can be used interchangeably with the term “non-coding”, and particularly refers to a nucleic acid sequence in the absence of a peptide or protein coding portion, such as a plasmid vector without a gene insert. The phrase “operatively linked” refers to linking a nucleic acid molecule to a transcription control sequence in a manner such that the molecule is capable of expression when transfected (i.e., transformed, transduced or transfected) into a host cell. Therefore, a nucleic acid sequence that is “not operatively linked to a transcription control sequence” refers to any nucleic acid sequence, including both coding and non-coding nucleic acid sequences, which are not linked to a transcription control sequence in a manner such that the molecule is capable of expression when transfected into a host cell. It is noted that this phrase does not preclude the presence of a transcription control sequence in the nucleic acid molecule.

In some embodiments of the present invention, a nucleic acid sequence included in a therapeutic composition of the present invention is incorporated into a recombinant nucleic acid molecule, and encodes an immunogen and/or a cytokine. As discussed in detail below, preferred immunogens include a tumor antigen, an allergen or an antigen from an infectious disease pathogen (i.e., a pathogen antigen). The phrase “recombinant molecule” primarily refers to a nucleic acid molecule or nucleic acid sequence operatively linked to a transcription control sequence, but can be used interchangeably with the phrase “nucleic acid molecule” which is administered to a mammal.

According to the present invention, an isolated, or biologically pure, nucleic acid molecule or nucleic acid sequence, is a nucleic acid molecule or sequence that has been removed from its natural milieu. As such, “isolated” and “biologically pure” do not necessarily reflect the extent to which the nucleic acid molecule has been purified. An isolated nucleic acid molecule useful in the present composition can include DNA, RNA, or derivatives of either DNA or RNA. Typically, an oligonucleotide has a nucleic acid sequence from about 1 to about 500 nucleotides, and more typically, is at least about 5 nucleotides in length, or any length increasing by whole integers (e.g., 6, 7, 8, 9, 10 and so on), up to about 500 nucleotides. In a preferred embodiment, an oligonucleotide for use in the present invention includes an oligonucleotide containing a cytosine-guanine (CpG) motif that is immunogenic in a mammal. In another embodiment, the oligonucleotide (for example CpG) is demethylated. Methylation of CpG motifs in DNA is involved in the control of gene expression and in several other epigenic effects. It suppresses the immuno-stimulation properties of bacterial or viral DNAs that contain CpGs. It is further known in the art that bacterial DNA and synthetic oligodeoxynucleotides containing unmethylated CpG-motifs in a particular sequence context can activate vertebrate immune cells.

Immune activation by PRRL:nucleic acid:lipid complexes of the present invention can be induced by eukaryotic as well as prokaryotic nucleic acids, indicating that there is some property of the PRRL:nucleic acid:lipid complexes that is inherently immune activating, regardless of the source of the nucleic acids. Therefore, the nucleic acid molecule can be derived from any source, including mammalian, bacterial, insect, or viral sources, since the present inventors have discovered that the source of the nucleic acid does not have a significant effect on the ability to elicit an immune response by the nucleic acid-lipid complex. In one embodiment of the present invention, the nucleic acid molecule used in a therapeutic composition of the present invention is not a bacterial nucleic acid molecule.

An isolated immunogen-encoding (e.g., a tumor antigen-, allergen-, or pathogen antigen-) or cytokine-encoding nucleic acid molecule can be obtained from its natural source, either as an entire (i.e., complete) gene or a portion thereof capable of encoding: a tumor antigen protein having a Becell and/or T cell epitope, an allergen having a B cell and/or T cell epitope, a pathogen antigen having a B cell and/or a T cell epitope, or a cytokine protein capable of binding to a complementary cytokine receptor. A nucleic acid molecule can also be produced using recombinant DNA technology (e.g., polymerase chain reaction (PCR) amplification, cloning) or chemical synthesis. Nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications do not substantially interfere with the nucleic acid molecule's ability to encode an immunogen or a cytokine useful in the method of the present invention.

A nucleic acid molecule homologue can be produced using a number of methods known to those skilled in the art (see, for example, Sambrook, et al., Molecular Cloning: A Laboratory Manual, Cold Spring Harbor Labs Press, 1989), which is incorporated herein by reference in its entirety. For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid (e.g., tumor antigen, allergen or pathogen antigen immunogenicity, or cytokine activity, as appropriate). Techniques to screen for immunogenicity, such as tumor antigen, allergen or pathogen antigen immunogenicity, or cytokine activity, are known to those of skill in the art and include a variety of in vitro and in vivo assays.

As heretofore disclosed, immunogen or cytokine proteins of the present invention include, but are not limited to, proteins encoded by nucleic acid molecules having full-length immunogen or cytokine coding regions; proteins encoded by nucleic acid molecules having partial immunogen regions which contain at least one T cell epitope and/or at least one B cell epitope; proteins encoded by nucleic acid molecules having cytokine coding regions capable of binding to a complementary cytokine receptor; fusion proteins; and chimeric proteins comprising combinations of different immunogens and/or cytokines.

One embodiment of the present invention includes an isolated nucleic acid molecule that encodes at least a portion of a full-length immunogen, including a tumor antigen, allergen or pathogen antigen, or a homologue of such immunogens. As used herein, “at least a portion of an immunogen” refers to a portion of an immunogen protein containing a T cell and/or a B cell epitope. In one embodiment, an immunogen-encoding nucleic acid molecule includes an entire coding region of such an immunogen. As used herein, a homologue of an immunogen is a protein; having an amino acid sequence that is sufficiently similar to a natural immunogen amino acid sequence (i.e., a naturally occurring, endogenous, or wild-type immunogen) that a nucleic acid sequence encoding the homologue encodes a protein capable of eliciting an immune response against the natural immunogen.

A tumor antigen-encoding nucleic acid molecule of the present invention encodes an antigen that can include tumor antigens having epitopes that are recognized by T cells, tumor antigens having epitopes that are recognized by B cells, tumor antigens that are exclusively expressed by tumor cells, and tumor antigens that are expressed by tumor cells and by non-tumor cells. Preferably, tumor antigens useful in the present method have at least one T cell and/or B cell epitope. Therefore, expression of the tumor antigen in a tissue of a mammal elicits a tumor antigen-specific immune response against the tumor in the tissue of the mammal. As discussed above, the present inventors have found that administration of the nucleic acid:lipid complex of the present invention elicits a strong, systemic, non-antigen-specific, anti-tumor response in vivo, and this effect enhances the antigen-specific immune response to a tumor antigen expressed by the nucleic acid molecule.

In a preferred embodiment, a nucleic acid molecule of the present invention encodes a tumor antigen from a cancer selected from the group of melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, primary hepatic cancers, lung cancers, pancreatic cancers, gastrointestinal cancers, renal cell carcinomas, hematopoietic neoplasias and metastatic cancers thereof.

According to the present invention, a pathogen antigen-encoding nucleic acid molecule of the present invention encodes an antigen from an infectious disease pathogen that can include pathogen antigens having epitopes that are recognized by T cells, pathogen antigens having epitopes that are recognized by B cells, pathogen antigens that are exclusively expressed by pathogens, and pathogen antigens that are expressed by pathogens and by other cells. Preferably, pathogen antigens useful in the present method have at least one T cell and/or B cell epitope and are exclusively expressed by pathogens (i.e., and not by the endogenous tissues of the infected mammal). Therefore, expression of the pathogen antigen in a tissue of a mammal elicits an antigen-specific immune response against the pathogen in the tissues of the mammal as well as systemically.

According to the present invention, a pathogen immunogen includes, but is not limited to, an immunogen that is expressed by a bacterium, a virus, a parasite, a prion, a rickettsial or a fungus. Preferred pathogen immunogens for use in the method of the present invention include immunogens which cause a chronic or an acute infectious disease in a mammal. For example, some preferred pathogen immunogens for use in the present method are immunogens from pathogens that cause chronic infections, including, but not limited to, immunodeficiency virus (HIV), Mycobacterium tuberculosis, herpesvirus, papillomavirus, Leishmania, Toxoplasma, Cryptococcus, Blastomyces, Histoplasma, and Candida. Also included would be antibiotic resistant strains of bacteria that can cause chronic infections, such as Staphylococcus, Pseudomonas, Streptococcus, Enterococcus, and Salmonella. Additionally, for immunization against acute disease, preferred pathogens from which immunogens can be derived include, but are not limited to, Bacillus anthracis, Francisella, Yersenia, Pasteurella, small pox, and other gram negative and gram positive bacterial pathogens.

In another embodiment of the present invention, a pathogen antigen for use in the method or composition of the present invention includes an immunogen from a virus. As discussed above, the present inventors have found that the composition and method of the present invention are particularly useful in the treatment of and protection against viral infections. Specifically, a nucleic acid may be further complexed to the PRRL:lipid complex when administered by a method of the present invention elicits a strong, systemic, non-antigen-specific, anti-viral response in vivo, regardless of whether or not the nucleic acid encodes or expresses an immunogen. When the nucleic acid sequence does encode a viral antigen that is operatively linked to a transcription control sequence such that the viral antigen is expressed in a tissue of a mammal, the present composition further elicits a strong, viral antigen-specific immune response in addition to the above-described systemic immune response. In a preferred embodiment, the immunogen may be from a virus selected from human immunodeficiency virus and feline immunodeficiency virus.

Another embodiment of the present invention includes an allergen-encoding nucleic acid molecule that encodes at least a portion of a full-length allergen or a homologue of the allergen protein, and includes allergens having epitopes that are recognized by T cells, allergens having epitopes that are recognized by B cells, and allergens that are a sensitizing agent in diseases associated with allergic inflammation. The allergens may be inhaled, cutaneous or oral. Preferred allergens to use in the therapeutic composition of the present invention include plant pollens, drugs, foods, venoms, insect excretions, molds, animal fluids, animal hair and animal dander.

Another embodiment of the present invention includes a cytokine-encoding nucleic acid molecule that encodes at least a portion of a full-length cytokine or a homologue of the cytokine protein. As used herein, “at least a portion of a cytokine” refers to a portion of a cytokine protein having cytokine activity and being capable of binding to a cytokine receptor. Preferably, a cytokine-encoding nucleic acid molecule includes an entire coding region of a cytokine. As used herein, a homologue of a cytokine is a protein having an amino acid sequence that is sufficiently similar to a natural cytokine amino acid sequence so as to have cytokine activity (i.e. activity associated with naturally occurring, or wild-type cytokines). In accordance with the present invention, a cytokine includes a protein that is capable of affecting the biological function of another cell. A biological function affected by a cytokine can include, but is not limited to, cell growth or arrest, cell differentiation or cell death. Preferably, a cytokine of the present invention is capable of binding to a specific receptor on the surface of a cell, thereby affecting the biological function of a cell.

A cytokine-encoding nucleic acid molecule of the present invention encodes a cytokine that is capable of affecting the biological function of a cell, including, but not limited to, a lymphocyte, a muscle cell, a hematopoietic precursor cell, a mast cell, a natural killer cell, a macrophage, a monocyte, an epithelial cell, an endothelial cell, a dendritic cell, a mesenchymal cell, a Langerhans cell, cells found in granulomas and tumor cells of any cellular origin, and more preferably a mesenchymal cell, an epithelial cell, an endothelial cell, a muscle cell, a macrophage, a monocyte, a T cell and a dendritic cell.

A preferred cytokine nucleic acid molecule of the present invention encodes a hematopoietic growth factor, an interleukin, an interferon, an immunoglobulin superfamily molecule, a tumor necrosis factor family molecule and/or a chemokine (i.e., a protein that regulates the migration and activation of cells, particularly phagocytic cells). A more preferred cytokine nucleic acid molecule of the present invention encodes an interleukin. An even more preferred cytokine nucleic acid molecule useful in the method of the present invention encodes interleukin-2 (IL-2), interleukin-7 (IL-7), interleukin-12 (IL-12), interleukin-15 (IL-15), interleukin-18 (IL-18), and/or interferon-γ (IFN-γ). A most preferred cytokine nucleic acid molecule useful in the method of the present invention encodes interleukin-2 (IL-2), interleukin-12 (IL-12), interleukin-18 (IL-18) and/or interferon-γ (IFN-γ).

As will be apparent to one of skill in the art, the present invention is intended to apply to cytokines derived from all types of mammals. A preferred mammal from which to derive cytokines includes a mouse, a human and a domestic pet (e.g., dog, cat). A more preferred mammal from which to derive cytokines includes a dog and a human. An even more preferred mammal from which to derive cytokines is a human.

According to the present invention, a cytokine-encoding nucleic acid molecule of the present invention is preferably derived from the same species of mammal as the mammal to be treated. For example, a cytokine-encoding nucleic acid molecule derived from a canine (i.e., dog) nucleic acid molecule is preferably used to treat a disease in a canine. The present invention includes a nucleic acid molecule of the present invention operatively linked to one or more transcription control sequences to form a recombinant molecule. As discussed above, the phrase “operatively linked” refers to linking a nucleic acid molecule to a transcription control sequence in a manner such that the molecule is capable of expression when transfected (i.e., transformed, transduced or transfected) into a host cell. Preferably, a nucleic acid molecule used in a composition of the present invention is operatively linked to a transcription control sequence that allows for transient expression of the molecule in the recipient mammal. To avoid adverse affects of prolonged immune activation (e.g., shock, excessive inflammation, immune tolerance), it is a preferred embodiment of the present invention that an immunogen or cytokine encoded by a nucleic acid molecule be expressed in the immunized mammal for about 72 hours to about 1 month, and preferably, from about 1 week to about 1 month, and more preferably, from about 2 weeks to about 1 month. Expression of a longer period of time than 1 month is not desired in instances where undesirable effects associated with prolonged immune activation occur. However, if such effects do not occur for a particular composition or can be avoided or controlled, then extended expression is acceptable. In one embodiment, transient expression can be achieved by selection of suitable transcription control sequences, for example. Transcription control sequences suitable for transient gene expression are discussed below.

Transcription control sequences are sequences that control the initiation, elongation, and termination of transcription. Particularly important transcription control sequences are those that control transcription initiation, such as promoter, enhancer, operator and repressor sequences. Suitable transcription control sequences include any transcription control sequence that can function in at least one of the recombinant cells useful in the method of the present invention. A variety of such transcription control sequences are known to those skilled in the art. Preferred transcription control sequences include those that function in mammalian, bacteria, insect cells, and preferably in mammalian cells. More preferred transcription control sequences include, but are not limited to, simian virus 40 (SV-40), β-actin, retroviral long terminal repeat (LTR), Rous sarcoma virus (RSV), cytomegalovirus (CMV), tac, lac, trp, trc, oxy-pro, omp/lpp, rrnB, bacteriophage lambda (λ) (such as λpL and λpR and fusions that include such promoters), bacteriophage T7, T7lac, bacteriophage T3, bacteriophage SP6, bacteriophage SP01, metallothionein, alpha mating factor, Pichia alcohol oxidase, alphavirus subgenomic promoters (such as Sindbis virus subgenomic promoters), baculovirus, Heliothis zea insect virus, vaccinia virus and other poxviruses, herpesvirus, and adenovirtis transcription control sequences, as well as other sequences capable of controlling gene expression in eukaryotic cells. Additional suitable transcription control sequences include tissue-specific promoters and enhancers (e.g., T cell-specific enhancers and promoters). Transcription control sequences of the present invention can also include naturally occurring transcription control sequences naturally associated with a gene encoding an immunogen, including tumor antigen, an allergen, a pathogen antigen or a cytokine.

Particularly preferred transcription control sequences for use in the present invention include promoters that allow for transient expression of a nucleic acid molecule that is to be expressed, thereby allowing for expression of the protein encoded by the nucleic acid molecule to be terminated after a time sufficient to elicit an immune response. Adverse effects related to prolonged activation of the immune system can be avoided by selection of promoters and other transcription control factors that allow for transient expression of a nucleic acid molecule. This is yet another point of difference between the method of the present invention and previously described gene therapy/gene replacement protocols. Suitable promoters for use with nucleic acid molecules encoding immunogens and/or cytokines for use in the present invention include cytomegalovirus (CMV) promoter and other non-retroviral virus-based promoters such as RSV promoters, adenovirus promoters and Simian virus promoters. LTR, tissue-specific promoters, promoters from self-replication viruses and papillomavirus promoters, which may be quite desirable in gene therapy/gene replacement protocols because they provide prolonged expression of a transgene, are not preferred transcription control sequences for use in the present invention.

Recombinant molecules of the present invention, which can be either DNA or RNA, can also contain additional regulatory sequences, such as translation regulatory sequences, origins of replication, and other regulatory sequences that are compatible with the recombinant cell. In one embodiment, a recombinant molecule of the present invention also contains secretory signals (i.e., signal segment nucleic acid sequences) to enable an expressed immunogen or cytokine protein to be secreted from the cell that produces the protein. Suitable signal segments include: (1) an immunogen signal segment (e.g., a tumor antigen, allergen or pathogen antigen signal segment); (2) a cytokine signal segment; (3) or any heterologous signal segment capable of directing the secretion of an immunogen and/or cytokine protein according to the present invention.

Preferred recombinant molecules of the present invention include a recombinant molecule containing a nucleic acid sequence encoding an immunogen, a recombinant molecule containing a nucleic acid sequence encoding a cytokine, or a recombinant molecule containing both a nucleic acid sequence encoding an immunogen and a nucleic acid sequence encoding a cytokine to form a chimeric recombinant molecule (i.e., the nucleic acid sequence encoding the immunogen and the nucleic acid sequence encoding the cytokine are in the same recombinant molecule). The nucleic acid molecules contained in such recombinant chirieric molecules are operatively linked to one or more transcription control sequences, in which each nucleic acid molecule contained in a chimeric recombinant molecule can be expressed using the same or different transcription control sequences.

One or more recombinant molecules of the present invention can be used to produce an encoded product (i.e., an immunogen protein or a cytokine protein) useful in the method of the present invention. In one embodiment, an encoded product is produced by expressing a nucleic acid molecule as described herein under conditions effective to produce the protein. A preferred method to produce an encoded protein is by transfecting a host cell with one or more recombinant molecules to form a recombinant cell. Suitable host cells to transfect include any mammalian cell that can be transfected. Host cells can be either untransfected cells or cells that are already transformed with at least one nucleic acid molecule. Host cells according to the present invention can be any cell capable of producing an immunogen (e.g., tumor, allergen or pathogen) and/or a cytokine according to the present invention. A preferred host cell includes a mammalian lung cells, lymphocytes, muscle cells, hematopoictic precursor cells, mast cells, natural killer cells, macrophages, monocytes, epithelial cells, endothelial cells, dendritic cells, mesenchymal cells, Langerhans cells, cells found in granulomas and tumor cells of any cellular origin. An even more preferred host cell of the present invention includes mammalian mesenchymal cells, epithelial cells, endothelial cells, macrophages, monocytes, lung cells, muscle cells, T cells and dendritic cells.

According to the method of the present invention, a host cell is preferably transfected in vivo (i.e., in a mammal) as a result of intravenous Or intraperitoneal administration to a mammal of a nucleic acid molecule complexed to a liposome delivery vehicle. Transfection of a nucleic acid molecule into a host cell according to the present invention can be accomplished by any method by which a nucleic acid molecule administered with a liposome delivery vehicle can be inserted into the cell in vivo, and includes lipofection.

It may be appreciated by one skilled in the art that use of recombinant DNA technologies may improve expression of transfected nucleic acid molecules by manipulating, for example, the duration of expression of the transgene (i.e., recombinant nucleic acid molecule), the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, increasing the duration of expression of the recombinant molecule, substitutions or modifications of transcription control signals (e.g., promoters, operators, enhancers), substitutions or modifications of translational control signals (e.g., ribosome binding sites, Shine-Dalgamo sequences), modification of nucleic acid molecules of the present invention to correspond to the codon usage of the host cell, and deletion of sequences that destabilize transcripts. The activity of an expressed recombinant protein of the present invention may be improved by fragmenting, modifying, or derivatizing nucleic acid molecules encoding such a protein. Additionally, a nucleic acid molecule, and particularly a plasmid portion, including transcription control sequences, can be modified to make the nucleic acids more immunostimulatory, such as by the addition of CpG moieties to the nucleic acids.

One embodiment of the method of the present invention, when the mammal has cancer, a therapeutic composition to be intravenously administered to the mammal comprises a plurality of recombinant nucleic acid molecules, wherein each of the recombinant nucleic acid molecules comprises a cDNA sequence, each of the cDNA sequences encoding a tumor antigen or a fragment thereof (i.e., at least a portion of a tumor antigen as defined above, preferably a portion containing a T or B cell epitope). The cDNA sequences are amplified from total RNA that has been isolated from an autologous tumor sample. Each of the plurality of cDNA sequences is operatively linked to a transcription control sequence. Administration of such a therapeutic composition to a mammal that has cancer results in the expression of the cDNA sequences encoding the tumor antigens in the tissue of the mammal (pulmonary tissue by intravenous administration and spleen and liver by intraperitoneal administration). In a further embodiment, such a therapeutic composition comprises a recombinant nucleic acid molecule having a nucleic acid sequence encoding a cytokine, wherein the nucleic acid sequence is operatively linked to a transcription control sequence. Administration of such a therapeutic composition to a mammal results in the expression of the nucleic acid sequence encoding the cytokine in the above-mentioned tissues of the mammal. According to this embodiment of the present invention, an autologous tumor sample is derived from the mammal to which the therapeutic composition is to be administered. Therefore, the cDNA sequences in the therapeutic composition will encode tumor antigens present in the cancer against which an immune response is to be elicited. In this embodiment, it is not necessary to know which of the antigens in a given tumor sample is the most immunogenic (i.e., the best immunogens), since substantially all of the antigens expressed by the tumor sample are administered to the mammal. In addition, eliciting an immune response against multiple tumor antigens/immunogens is likely to have the benefit of enhancing the therapeutic efficacy of the immune response against the cancer.

In this embodiment of the method of the present invention, a plurality of recombinant nucleic acid molecules as described can also be referred to as a library of nucleic acid molecules, and more particularly, a cDNA library. Methods to produce cDNA libraries are well known in the art. Such methods are disclosed, for example, in Sambrook, et al., supra. More particularly, in this embodiment, a therapeutic composition includes a plurality of recombinant cDNA molecules encoding tumor antigens, or fractions thereof, which represents the genes that are expressed by an autologous tumor sample. Such a plurality of recombinant nucleic acid molecules can be produced, for example by isolating total RNA from an autologous tumor sample, converting (i.e., amplifying) the RNA into a plurality of cDNA molecules, and then preparing a cDNA library by cloning the cDNA molecules into recombinant vectors to form a plurality of recombinant molecules. As used herein, total RNA refers to all of the RNA isolatable from a cellular sample using standard methods known in the art, and typically includes mRNA, hnRNA, tRNA and rRNA. Methods for isolating total RNA from a cellular sample, such as a tumor sample, are known in the art (See for example, Sambrook, et al., supra). In a further embodiment, prior to amplification of cDNA from the total RNA, the RNA can be selected to isolate poly-A RNA (i.e., RNA comprising a poly-A tail at the 3′ terminus, reflective of mRNA, the primary RNA transcript which encodes a protein expressed by a cell). In yet another embodiment, such a cDNA library can be “subtracted” against a cDNA library from a normal cellular sample in the mammal in order to remove nucleic acid molecules encoding antigens present in non-tumor cells (i.e., normal cells) of the mammal, thereby enriching the tumor-specific immune response against the tumor specific antigens and preventing deleterious immune responses. Methods for subtraction of a nucleic acid library are also known in the art (See Sambrook, et al., supra).

In yet another embodiment of the present invention of the method to elicit an immune response in a mammal that has cancer, a therapeutic composition to be intravenously or intraperitoneally administered to a mammal comprises a plurality of recombinant nucleic acid molecules, wherein each of the recombinant nucleic acid molecules comprises a cDNA sequence, each of the cDNA sequences encoding a tumor antigen or a fragment thereof (i.e., at least a portion of a tumor antigen as defined above). In this embodiment, the cDNA sequences are amplified from total RNA that has been isolated from a plurality of allogeneic tumor samples of the same histological tumor type. Each of the plurality of cDNA sequences is operatively linked to a transcription control sequence. Administration of such a therapeutic composition to a mammal that has cancer results in the expression of the cDNA sequences encoding the tumor antigens in the tissue of the mammal (according to the route of administration, as previously discussed). In a further embodiment, such a therapeutic composition comprises a recombinant nucleic acid molecule having a nucleic acid sequence encoding a cytokine, wherein the nucleic acid sequence is operatively linked to a transcription control sequence. Administration of such a therapeutic composition to a mammal results in the expression of the nucleic acid sequence encoding the cytokine in the tissues of the mammal.

In this embodiment of the present invention, a plurality of recombinant nucleic acid molecules comprising cDNA sequences encoding tumor antigens (i.e., a cDNA library) is prepared from the total RNA isolated from a plurality of allogeneic tumor samples of the same histological tumor type. According to the present invention, a plurality of allogeneic tumor samples are tumor samples of the same histological tumor type, isolated from two or more mammals of the same species who differ genetically at least within the major histocompatibility complex (MHC), and typically at other genetic, loci. Therefore, the plurality of recombinant molecules encoding tumor antigens is representative of the substantially all of the tumor antigens present in any of the individuals from which the RNA was isolated. This embodiment of the method of the present invention provides a genetic vaccine which compensates for natural variations between individual patients in the expression of tumor antigens from tumors of the same histological tumor type. Therefore, administration of this therapeutic composition is effective to elicit an immune response against a variety of tumor antigens such that the same therapeutic composition can be administered to a variety of different individuals. Such a therapeutic composition delivered by the present method is particularly useful as a treatment, but may also be useful as a preventative (i.e., prophylactic) therapy. Methods to prepare such a cDNA library from a plurality of allogeneic tumor samples are the same as those described above for autologous tumor samples.

In yet another embodiment of the present invention of the method to elicit an immune response in a mammal, a therapeutic composition to be intravenously or intraperitoneally administered to a mammal comprises a plurality of recombinant nucleic acid molecules, wherein each of the recombinant nucleic acid molecules comprises a cDNA sequence, each of the cDNA sequences encoding an immunogen from an infectious disease pathogen or a fragment thereof (i. e., at least a portion of a pathogen antigen as defined above). In this embodiment, the cDNA sequences are amplified from total RNA that has been isolated from an infectious disease pathogen. Each of the plurality of cDNA sequences is operatively linked to a transcription control sequence. Administration of such a therapeutic composition to a mammal that has or might contract an infectious disease results in the expression of the cDNA sequences encoding the pathogen antigens in the tissue of the mammal (according to the route of administration, as previously discussed). In a further embodiment, such a therapeutic composition comprises a recombinant nucleic acid molecule having a nucleic acid sequence encoding a cytokine, wherein the nucleic acid sequence is operatively linked to a transcription control sequence. Administration of such a therapeutic composition to a mammal results in the expression of the nucleic acid sequence encoding the cytokine in the tissues of the mammal.

In this embodiment of the present invention, the plurality of recombinant molecules encoding pathogen antigens is representative of the substantially all of the antigens present in the infectious disease pathogen from which the RNA was isolated. In this embodiment, it is not necessary to know which of the antigens in a given pathogen is the most immunogenic (i.e., the best immunogens), since substantially all of the antigens expressed by the pathogen are administered to the mammal. In addition, eliciting an immune response against multiple pathogen antigens/immunogens is likely to have the benefit of enhancing the therapeutic efficacy of the immune response against the infectious disease. Methods to prepare such a cDNA library from an infectious disease pathogen are the same as those described above for tumor samples.

In yet another embodiment of the present invention of the method to elicit an immune response in a mammal, a therapeutic composition to be intravenously or intraperitoneally administered to a mammal comprises a plurality of recombinant nucleic acid molecules, each of the recombinant nucleic acid molecules comprising a cDNA sequence amplified from total RNA isolated from at least one allergen. In this embodiment, the cDNA sequences are amplified from total RNA, or a fragment thereof, that has been isolated from at least one, and preferably, multiple, allergens. Each of the plurality of cDNA sequences is operatively linked to a transcription control sequence. Administration of such a therapeutic composition to a mammal that has or might contract a disease associated with allergic inflammation results in the expression of the cDNA sequences encoding the allergens in the tissue of the mammal (according to the route of administration, as previously discussed). In a further embodiment, such a therapeutic composition comprises a recombinant nucleic acid molecule having a nucleic acid sequence encoding a cytokine, wherein the nucleic acid sequence is operatively linked to a transcription control sequence. Administration of such a therapeutic composition to a mammal results in the expression of the nucleic acid sequence encoding the cytokine in the tissues of the mammal. In this embodiment of the present invention, the plurality of recombinant molecules encoding allergens is representative of the substantially all of the epitopes present in the allergen from which the RNA was isolated. Additionally, more than one allergen can be administered simultaneously.

Another embodiment of the present invention relates to a method to elicit a tumor antigen-specific immune response and a systemic, non-specific immune response in a mammal that has cancer, which includes the step of intravenously or intraperitoneally administering to the mammal a therapeutic composition which includes: (a) a liposome delivery vehicle; (b) at least one PRRL; and (c) total RNA isolated from a tumor sample, wherein the RNA encodes tumor antigens or fragments thereof. Administration of such a therapeutic composition to the mammal results in the expression of the RNA encoding tumor antigens or fragments thereof in the tissue of the mammal. In a preferred embodiment, the RNA is enriched for poly-A RNA prior to administration of the therapeutic composition to the mammal, as described above. In a further embodiment, the therapeutic composition comprises a recombinant nucleic acid molecule having a nucleic acid sequence encoding a cytokine, wherein the nucleic acid sequence is operatively linked to a transcription control sequence. Administration of such a therapeutic composition to a mammal results in expression of the nucleic acid sequence encoding the cytokine in the tissue of the mammal to which it is directed.

In this embodiment of the present invention, total RNA or more preferably, poly-A enriched RNA, is isolated from a tumor sample as previously described (See Sambrook, et al., supra.), complexed with a liposome delivery vehicle and administered intravenously or intraperitoneally to a mammal that has cancer. The RNA encoding substantially all of the tumor antigens of the tumor sample is then expressed in the tissues of the mammal. Although RNA is normally degraded rapidly in serum by RNAses, the present inventors believe that RNA complexed to cationic lipids is protected from such RNAses until it reaches the tissues, where gene expression occurs. The advantage of administering RNA directly to a mammal according to this particular embodiment of the method of the present invention is that an immune response can be elicited against multiple tumor antigens directly in vivo, without requiring any substantial in vitro manipulations of the tumor tissues or host immune cells.

Another embodiment of the present invention relates to a method to elicit a pathogen antigen-specific immune response and a systemic, non-specific immune response in a mammal that has an infectious disease, which includes the step of intravenously or intraperitoneally administering to the mammal a therapeutic composition which includes: (a) a liposome delivery vehicle; (b) at least one PRRL; and (c) total RNA isolated from an infectious disease pathogen, wherein the RNA encodes pathogen antigens or fragments thereof. Administration of such a therapeutic composition to the mammal results in the expression of the RNA encoding pathogen antigens or fragments thereof in the tissue of the mammal. In a preferred embodiment, the RNA is enriched for poly-A RNA prior to administration of the therapeutic composition to the mammal, as described above. In a further embodiment, the therapeutic composition comprises a recombinant nucleic acid molecule having a nucleic acid sequence encoding a cytokine, wherein the nucleic acid sequence is operatively linked to a transcription control sequence. Administration of such a therapeutic composition to a mammal results in expression of the nucleic acid sequence encoding the cytokine in the tissue of the mammal.

Another embodiment of the present invention relates to a method to elicit an allergen-specific immune response and a systemic, non-specific immune response in a mammal that has a disease associated with allergic inflammation, which includes the step of intravenously or intraperitoneally administering to the mammal a therapeutic composition which includes: (a) a liposome delivery vehicle; (b) at least one PkRL and (c) total RNA isolated from an allergen, wherein the RNA encodes at least one allergen protein or a fragment thereof. Administration of such a therapeutic composition to the mammal results in the expression of the RNA encoding at least one allergen or a fragment thereof in the tissue of the mammal. In a preferred embodiment, the RNA is enriched for poly-A RNA prior to administration of the therapeutic composition to the mammal, as described above. In a further embodiment, the therapeutic composition comprises a recombinant nucleic acid molecule having a nucleic acid sequence encoding a cytokine, wherein the nucleic acid sequence is operatively linked to a transcription control sequence. Administration of such a therapeutic composition to a mammal results in expression of the nucleic acid sequence encoding the cytokine in the tissue of the mammal.

A therapeutic Composition of the present invention includes a liposome delivery vehicle. According to the present invention, a liposome delivery vehicle comprises a lipid composition that is capable of preferentially delivering a therapeutic composition of the present invention to the pulmonary tissues in a mammal when administration is intravenous, and to the spleen and liver tissues of a mammal when administration is intraperitoneal. The phrase “preferentially delivering” means that although the liposome can deliver a nucleic acid molecule to sites other than the pulmonary or spleen and liver tissue of the mammal, these tissues are the primary site of delivery.

A liposome delivery vehicle of the present invention can be modified to target a particular site in a mammal, thereby targeting and making use of a PRRL and/or a nucleic acid molecule of the present invention at that site. Suitable modifications include manipulating the chemical formula of the lipid portion of the delivery vehicle. Manipulating the chemical formula of the lipid portion of the delivery vehicle can elicit the extracellular or intracellular targeting of the delivery vehicle. For example, a chemical can be added to the lipid formula of a liposome that alters the charge of the lipid bilayer of the liposome so that the liposome fuses with particular cells having particular charge characteristics. Other targeting mechanisms, such as targeting by addition of exogenous targeting molecules to a liposome (i.e., antibodies) are not a necessary component (but may be an optional component) of the liposome delivery vehicle of the present invention, since effective immune activation at immunologically active organs is already provided by the composition and route of delivery of the present compositions without the aid of additional targeting mechanisms. Additionally, for efficacy, the present invention does not require that a protein encoded by a given nucleic acid molecule be expressed within the target cell (e.g., tumor cell, pathogen, etc.). The compositions and method of the present invention are efficacious when the proteins are expressed in the vicinity of (i.e., adjacent to) the target site, including when the proteins are expressed by non-target cells.

A liposome delivery vehicle is preferably capable of remaining stable in a mammal for a sufficient amount of time to deliver the PRRL or the PRRL and a nucleic acid molecule of the present invention to a preferred site in the mammal. A liposome delivery vehicle of the present invention is preferably stable in the mammal into which it has been administered for at least about 30 minutes, more preferably for at least about 1 hour and even more preferably for at least about 24 hours.

A liposome delivery vehicle of the present invention comprises a lipid composition that is capable of fusing with the plasma membrane of the targeted cell to deliver a PRRL and if desired a nucleic acid molecule into a cell. Preferably, when a PRRL:liposome complex of the present invention is administered intravenously, the transfection efficiency of the PRRL:liposome complex of the present invention is at least about 1 picogram (pg) of protein expressed per milligram (mg) of total tissue protein per microgram (μg) of nucleic acid delivered. More preferably, the transfection efficiency of a PRRL:liposome complex of the present invention is at least about 10 pg of protein expressed per mg of total tissue protein per μg of nucleic acid delivered; and even more preferably, at least about 50 pg of protein expressed per mg of total tissue protein per μg of nucleic acid delivered; and most preferably, at least about 100 pg of protein expressed per mg of total tissue protein per ug of nucleic acid delivered. When the route of delivery of a PRRL:lipid complex of the present invention is intraperitoneal, the transfection efficiency of the complex can be as low as 1 fg of protein expressed per mg of total tissue protein per μg of nucleic acid delivered, with the above amounts being more preferred.

A preferred liposome delivery vehicle of the present invention is between about 100 and 500 nanometers (nm), more preferably between about 150 and 450 nm and even more preferably between about 200 and 400 nm in diameter.

Suitable liposomes for use with the present invention include any liposome. Preferred liposomes of the present invention include those liposomes commonly used in, for example, gene delivery methods known to those of skill in the art. Preferred liposome delivery vehicles comprise multilamellar vesicle (MLV) lipids and extruded lipids. Methods for preparation of MLV's are well known in the art and are described, for example, in the Examples section. According to the present invention, “extruded lipids” are lipids which are prepared similarly to MLV lipids, but which are subsequently extruded through filters of decreasing size, as described in Templeton, et al., Nature Biotech., 15:647-652(1997), which is incorporated herein by reference in its entirety. Although small unilamellar vesicle (SUV) lipids can be used in the composition and method of the present invention, the present inventors have found that multilamellar vesicle lipids are significantly more immunostimulatory than SUVs when complexed with nucleic acids in vivo. More preferred liposome delivery vehicles comprise liposomes having a polycationic lipid composition (i.e., cationic liposomes) and/or liposomes having a cholesterol backbone conjugated to polyethylene glycol. Preferred cationic liposome compositions include, but are not limited to DOTMA and cholesterol, DOTAP and cholesterol, DOTIM and cholesterol, and DDAB and cholesterol. A most preferred liposome composition for use as a delivery vehicle in the method of the present invention includes DOTAP and cholesterol.

Complexing a liposome with a PRRL of the present invention can be achieved using methods standard in the art (see, for example, methods in U.S. patent application Ser. No. 09/104,759). See, Example 16.

According to the present invention a cationic lipid:PRRL complex is also referred to herein as a TLAC. A cationic lipid:DNA complex, wherein the DNA is an empty vector can be referred to as EV/CLDC. A CLDC that is further complexed with an immunogen according to the present invention can be referred to as a lipid-antigen-DNA complex (LADC) or as a vaccine (Vacc) or therapeutic composition.

A suitable concentration of PRRL of the present invention to add to a liposome includes a concentration effective for delivering a sufficient amount of PRRL into a mammal such that a systemic immune response is elicited. While a suitable concentration of nucleic acid molecule if added to the present invention includes a concentration effective for delivering a sufficient amount of nucleic acid molecule into a mammal such that a systemic immune response is elicited. When the nucleic acid molecule encodes an immunogen or a cytokine, a suitable concentration of nucleic acid molecule to add to a liposome includes a concentration effective for delivering a sufficient amount of nucleic acid molecule into a cell such that the cell can produce sufficient immunogen and/or cytokine protein to regulate effector cell immunity in a desired manner. Preferably, from about 0.1 μg to about 10 μg of nucleic acid molecule of the present invention is combined with about 8 nmol liposomes, more preferably from about 0.5 μg to about 5 μg of nucleic acid molecule is combined with about 8 nmol liposomes, and even more preferably about 1.0 μg of nucleic acid molecule is combined with about 8 nmol liposomes. In one embodiment, the ratio of nucleic acids to lipids (μg nucleic acid:nmol lipids) in a composition of the present invention is preferably at least about 1:1 nucleic acid:lipid by weight (i.e., 1 μg nucleic acid:1 nmol lipid), and more preferably, at least about 1:5, and more preferably at least about 1:10, and even more preferably at least about 1:20. Ratios expressed herein are based on the amount of cationic lipid in the composition, and not on the total amount of lipid in the composition. In another embodiment, the ratio of nucleic acids to lipids in a composition of the present invention is preferably from about 1:1 to about 1:64 nucleic acid:lipid by weight; and more preferably, from about 1:5 to about 1:50 nucleic acid:lipid by weight; and even more preferably, from about 1:10 to about 1:40 nucleic acid:lipid by weight; and even more preferably, from about 1:15 to about 1:30 nucleic acid:lipid by weight. Another particularly preferred ratio of nucleic acid:lipid is from about 1:8 to 1:16, with 1:8 to 1:32 being more preferred. Typically, while non-systemic routes of nucleic acid administration (i.e., intramuscular, intratracheal, intradermal) would use a ratio of about 1:1 to about 1:3, systemic routes of administration according to the present invention can use much less nucleic acid as compared to lipid and achieve equivalent or better results than non-systemic routes. Moreover, compositions designed for gene therapy/gene replacement, even when administered by intravenous administration, typically use more nucleic acid (e.g., from 6:1 to 1:10, with 1:10 being the least amount of DNA used) as compared to the systemic immune activation composition and method of the present invention.

According to the present invention, an effective administration protocol (i.e., administering a therapeutic composition in an effective manner) comprises suitable dose parameters and modes of administration that result in elicitation of an immune response in a mammal that has a disease, preferably so that the mammal is protected from the disease. Effective dose parameters can be determined using methods standard in the art for a particular disease. Such methods include, for example, determination of survival rates, side effects (i.e., toxicity) and progression or regression of disease. In particular, the effectiveness of dose parameters of a therapeutic composition of the present invention when treating cancer can be determined by assessing response rates. Such response rates refer to the percentage of treated patients in a population of patients that respond with either partial or complete remission. Remission can be determined by, for example, measuring tumor size or microscopic examination for the presence of cancer cells in a tissue sample.

In accordance with the present invention, a suitable single dose size is a dose that is capable of eliciting an immune response in a mammal with a disease when administered one or more times over a suitable time period. Doses can vary depending upon the disease being treated. In the treatment of cancer, a suitable single dose can be dependent upon whether the cancer being treated is a primary tumor or a metastatic form of cancer. Doses of a therapeutic composition of the present invention suitable for use with intravenous or intraperitoneal administration techniques can be used by one of skill in the art to determine appropriate single dose sizes for systemic administration based on the size of a mammal.

In a preferred embodiment, an appropriate single dose of a PRRL:liposome or PRRL:nucleic acid:liposome complex of the present invention is from about 0.1 μg to about 100 μg per kg body weight of the mammal to which the complex is being administered. In another embodiment, an appropriate single dose is from about 1 μg to about 10 μg per kg body weight. In another embodiment, an appropriate single dose of PRRL:lipid complex is at least about 0.1 μg of PRRL to the mammal, more preferably at least about 1 μg of PRRL, even more preferably at least about 10 μg of PRRL, even more preferably at least about 50 μg of PRRL, and even more preferably at least about 100 μg of PRRL to the mammal.

Preferably, when PRRL:nucleic acid:liposome complex of the present invention contains a nucleic acid molecule which is to be expressed in the mammal, an appropriate single dose of a PRRL:nucleic acid:liposome complex of the present invention results in at least about 1 pg of protein expressed per mg of total tissue protein per μg of nucleic acid delivered. More preferably, an appropriate single dose of a nucleic acid:liposome complex of the present invention is a dose which results in at least about 10 pg of protein expressed per mg of total tissue protein per μg of nucleic acid delivered; and even more preferably, at least about 50 μpg of protein expressed per mg of total tissue protein per pg of nucleic acid delivered; and most preferably, at least about 100 pg of protein expressed per mg of total tissue protein per μg of nucleic acid delivered. When the route of delivery of a PRRL:lipid complex of the present invention is intraperitoneal, an appropriate single dose of a PRRL:liposome complex of the present invention is a dose which results in as low as 1 fg of protein expressed per mg of total tissue protein per μg of PRRL delivered, with the above amounts being more preferred.

A suitable single dose of a therapeutic composition of the present invention to elicit a systemic, non-antigen-specific immune response in a mammal is a sufficient amount of a nucleic acid molecule complexed to a liposome delivery vehicle, when administered intravenously or intraperitoneally, to elicit a cellular and/or humoral immune response in vivo in a mammal, as compared to a mammal which has not been administered with the therapeutic composition of the present invention (i.e., a control mammal). Preferred dosages of nucleic acid molecules to be included in a nucleic acid:lipid complex of the present invention have been discussed above.

A suitable single dose of a therapeutic composition to elicit an immune response against a tumor is a sufficient amount of a tumor antigen-encoding recombinant molecule, alone or in combination with a cytokine-encoding recombinant molecule, to reduce, and preferably eliminate, the tumor following lipofection of the recombinant molecules into cells of the tissue of the mammal that has cancer.

According to the present invention, a single dose of a therapeutic composition useful to elicit an immune response against an infectious disease and/or against a lesion associated with such a disease, comprising a pathogen-encoding recombinant molecule combined with liposomes, alone or in combination with a cytokine-encoding recombinant molecule with liposomes, is substantially similar to those doses used to treat a tumor (as described in detail above). Similarly, a single dose of a therapeutic composition useful to elicit an immune response against an allergen, comprising an allergen-encoding recombinant molecule combined with liposomes, alone or in combination with a cytokine-encoding recombinant molecule with liposomes, is substantially similar to those doses used to treat a tumor.

It will be obvious to one of skill in the art that the number of doses administered to a mammal is dependent upon the extent of the disease and the response of an individual patient to the treatment. For example, a large tumor may require more doses than a smaller tumor. In some cases, however, a patient having a large tumor may require fewer doses than a patient with a smaller tumor, if the patient with the large tumor responds more favorably to the therapeutic composition than the patient with the smaller tumor. Thus, it is within the scope of the present invention that a suitable number of doses includes any number required to treat a given disease.

It is to be noted that the method of the present invention further differs from previously described gene therapy/gene replacement protocols, because the time between administration and boosting of the PRRL:lipid complex is significantly longer than the typical administration protocol for gene therapy/gene replacement. For example, elicitation of an immune response using the compositions and methods of the present invention typically includes an initial administration of the therapeutic composition, followed by booster immunizations at 3-4 weeks after the initial administration, optionally followed by subsequent booster immunizations every 3-4 weeks after the first booster, as needed to treat a disease according to the present invention. In contrast, gene therapy/gene replacement protocols typically require more frequent administration of a nucleic acid in order to obtain sufficient gene expression to generate or replace the desired gene function (e.g., weekly administrations).

A preferred number of doses of a therapeutic composition comprising a PRRL, a tumor antigen-encoding recombinant molecule, alone or in combination with a cytokine-encoding recombinant molecule, complexed with a liposome delivery vehicle in order to elicit an immune response against a metastatic cancer, is from about 2 to about 10 administrations patient, more preferably from about 3 to about 8 administrations per patient, and even more preferably from about 3 to about 7 administrations per patient. Preferably, such administrations are given once every 3-4 weeks, as described above, until signs of remission appear, and then once a month until the disease is gone.

According to the present invention, the number of doses of a therapeutic composition to elicit an immune response against an infectious disease and/or a lesion associated with such disease, comprising a pathogen antigen-encoding recombinant molecule, alone or in combination with a cytokine-encoding recombinant molecule, complexed with a PRRL:liposome delivery vehicle, is substantially similar to those number of doses used to treat a tumor (as described in detail above).

A therapeutic composition is administered to a mammal in a fashion to elicit a systemic, non-antigen-specific immune response in a mammal, and when the nucleic acid molecule in the composition encodes an immunogen, to enable expression of the administered recombinant molecule of the present invention into an immunogenic protein (in the case of the tumor, pathogen antigen or allergen) or immunoregulatory protein (in the case of the cytokine) in the mammal to be treated for disease. According to the method of the present invention, a therapeutic composition is administered by intravenous or intraperitoneal injection, and preferably, intravenously. Intravenous injections can be performed using methods standard in the art. According to the method of the present invention, administration of the PRRL:nucleic acid:lipid complexes can be at any site in the mammal wherein systemic administration (i.e., intravenous or intraperitoneal administration) is possible, particularly when the liposome delivery vehicle comprises cationic liposomes. Administration at any site in a mammal will elicit a potent immune response when either intravenous or intraperitoneal administration is used, and particularly, when intravenous administration is used. Suitable sites for administration include sites in which the target site for immune activation is not restricted to the first organ having a capillary bed proximal to the site of administration (i.e., compositions can be administered at an administration site that is distal to the target immunization site). In other words, for example, intravenous administration of a composition of the present invention which is used to treat a kidney tumor in a mammal can be administered intravenously at any site in the mammal and will still elicit a strong anti-tumor immune response and be efficacious at reducing or eliminating the tumor, even though the kidney is not the first organ having a capillary bed proximal to the site of administration. When a specific anti-tumor effect is desired (i.e., reduction or elimination of a tumor) and the route of administration is intravenous, the site of administration again can be at any site by which a composition can be administered intravenously, regardless of the location of the tumor relative to the site of administration. For intraperitoneal administration with regard to anti-tumor efficacy (but not immune activation/immunization), it is preferable to use this mode of administration when the tumor is in the peritoneal cavity, or when the tumor is a small tumor. For immunization and immune activation, as discussed above, intraperitoneal administration is a suitable mode of administration, particularly in comparison to non-systemic routes, as demonstrated in the Examples section.

In the method of the present invention, therapeutic compositions can be administered to any member of the Vertebrate class, Mammalia, including, without limitation, primates, rodents, livestock and domestic pets. Livestock include mammals to be consumed or that produce useful products (e.g., sheep for wool production). Preferred mammals to protect include humans, dogs, cats, mice, rats, sheep, cattle, horses and pigs, with humans and dogs being particularly preferred, and humans being most preferred. While a therapeutic composition of the present invention is effective to elicit an immune response against a disease in inbred species of mammals, the composition is particularly useful for eliciting an immune response in outbred species of mammals.

As discussed above, a therapeutic composition of the present invention administered by the present method is useful for eliciting an immune response in a mammal having a variety of diseases, and particularly cancer, allergic inflammation and infectious diseases. A therapeutic composition of the present invention, when delivered intravenously or intraperitoneally, is advantageous for eliciting an immune response in a mammal that has cancer in that the composition overcomes the mechanisms by which cancer cells avoid immune elimination (i.e., by which cancer cells avoid the immune response effected by the mammal in response to the disease). Cancer cells can avoid immune elimination by, for example, being only slightly immunogenic, modulating cell surface antigens and inducing immune suppression. A suitable therapeutic composition for use in eliciting an immune response in a mammal that has cancer comprises a PRRL:lipid complex or alternatively a PRRL:nucleic acid:lipid complex of the present invention, wherein the nucleic acid either is not operatively linked to a transcription control sequence, or more preferably, encodes a tumor antigen-encoding recombinant molecule operatively linked to a transcription control sequence, alone or in combination with a cytokine-encoding recombinant molecule (separately or together). A therapeutic composition of the present invention, elicits a systemic, non-specific immune response in the mammal and, upon entering targeted pulmonary or spleen and liver cells, leads to the production of tumor antigen (and, in particular embodiments, cytokine protein) that activate cytotoxic T cells, natural killer cells, T helper cells and macrophages. Such cellular activation overcomes the otherwise relative lack of immune response to cancer cells, leading to the destruction of such cells.

A therapeutic composition of the present invention which may include a nucleic acid molecule encoding a tumor antigen is useful for eliciting an immune response in a mammal that has cancer, including both tumors and metastatic forms of cancer. Treatment with the therapeutic composition overcomes the disadvantages of traditional treatments for metastatic cancers. For example, compositions of the present invention can target dispersed metastatic cancer cells that cannot be treated using surgical methods. In addition, administration of such compositions do not result in the harmful side effects caused by many cancer therapies, such as, hyperthermia, photodynamic ultrasound, focused ultrasound, chemotherapy and radiation therapy, or surgery and can be administered repeatedly. Moreover, the compositions administered by the method of the present invention typically target the vesicles of tumors, so that expression of a tumor antigen or cytokine within the tumor cell itself is not necessary to provide efficacy against the tumor. Indeed, a general advantage of the present invention is that delivery of the composition itself elicits a powerful immune response and expression of the nucleic acid molecule at least in the vicinity of the target site (at or adjacent to the site) provides effective immune activation and efficacy against the target.

Alternatively, a cancer therapy, such as one or a combination of therapiea discussed above may be used in conjunction with the therapeutic compositions of the present invention. The rationale for combining a cancer therapy, such as radiation therapy, of the tumor with injection of the therapeutic composition of the present invention is to supply a source of tumor antigens for incorporation into the vaccine. Tumor irradiation triggers tumor cell apoptosis, will results in the release of free tumor antigens locally into the tumor tissues. When TLRC are injected into a tumor undergoing apoptosis, the tumor antigens released from the dying cells will spontaneously become incorporated into the TLRC (by virtue of charge-charge interactions), resulting in the in situ production of an autologous tumor vaccine. The tumor antigens incorporated into the TLRC adjuvant will then induce activation of local innate immunity, recruitment of professional antigen presenting cells (APC), followed by antigen uptake and presentation in the nearest draining lymph nodes. The injection of the TLRC would follow the delivery of radiation therapy. In this scenario, the immune effector cells would not be activated until the tumor antigens reached the draining lymph nodes and would thus be spared destruction when the tumor was irradiated again. Thus, the tumor could receive multiple cycles of radiation therapy and intratumoral TLRC delivery. This would serve as a booster vaccine for the immune system and further augment the induction of antitumor immunity. In addition, patients could still continue to receive the current standard treatment for their tumors, with no interruption in radiation scheduling. The cancer therapy could be administered prior to, concurrently with or following introduction of the composition of the present invention.

Other methods of inducing tumor cell apoptosis could also be combined with the local intratumoral injection of the TLRC adjuvant. For example, injection of pro-apoptotic drugs (eg, camptothecin), injection of photosensitizers together with UV exposure of the tumor, tumor electroporation, or local hyperthermia could all be used to elicit tumor cell apoptosis or necrosis with liberation of tumor antigens for incorporation into the TLRC adjuvant.

The current invention would be designed for treatment of any tumor that was accessible to both needle injection and radiation therapy. The proposed treatment schedule would be designed around standard radiation therapy protocols, which typically involve administration of multiple fractions of radiation locally to the tumor on a 5 day per week schedule, for 3-4 weeks. The proposed combination schedule would involve intratumoral injection of TLRC at the start of radiation therapy (day 1), and again on day 5, day 12, day 19 and possibly also on day 26. The TLRC injections into the tumor site would then continue on a twice per month basis for the next 3-4 months, or until either surgical tumor excision or tumor regression. The dose of TLRC/LNAC to be administered would be based on tumor size, and in the use of LNAC would typically be 250 to 1000 μg nucleic acid (either non-coding plasmid DNA or CpG oligonucleotides) per injection.

Typical tumors that could be treated by such an approach would include non-resectable head and neck tumors (eg, squamous cell carcinoma), recurrent melanomas, breast cancers, and other malignant tumors of the skin or subcutaneous tissues.

In another emodiment, a method for induction of antitumor immunity by combining intratumoral injection of TLRC with inducers of tumor apoptosis or necrosis is contemplated. This treatment approach would involve administration of agents (either by local or systemic injection) that elicited apoptosis or necrosis of tumor cells to provide a source of antigens for the TLRC adjuvant. The TLRC adjuvant would then be administered into the tumor tissues after administration of the apoptosis/necrosis agent. This would be repeated in a series of alternating cycles of apoptosis agent plus TLRC. Examples of such agents could include concurrent administration of photodynamic therapy and TLRC, inducers of apoptosis (FasL, TRAIL, camptothecin) or inducers of tumor necrosis or lysis, such as TNF-α or distilled water. Typical tumors that could be treated with such an approach include those listed above. In addition, with PDT, tumors in less accessible sites such as the liver or kidneys could be treated using ultrasound-guided injection to injection the TLRC into the tumor tissues.

A therapeutic composition of the present invention which includes a nucleic acid molecule encoding a tumor antigen is preferably used to elicit an immune response in a mammal that has a cancer which includes, but is not limited to, melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, primary hepatic cancers, liver cancers, lung cancers, pancreatic cancers, gastrointestinal cancers, renal cell carcinomas, hematopoietic neoplasias, cancers of mesenchymal tissues, and metastatic cancers thereof. Particularly preferred cancers to treat with a therapeutic composition of the present invention include primary lung cancers and pulmonary metastatic cancers. A therapeutic composition of the present invention is useful for eliciting an immune response in a mammal to treat tumors that can form in such cancers, including malignant and benign tumors. Preferably, expression of the tumor antigen in a pulmonary tissue of a mammal that has cancer (i.e., by intravenous delivery) produces a result selected from the group of alleviation of the cancer, reduction of a tumor associated with the cancer, elimination of a tumor associated with the cancer, prevention of metastatic cancer, prevention of the cancer and stimulation of effector cell immunity against the cancer.

A therapeutic composition of the present invention which includes a nucleic acid molecule encoding an immunogen from an infectious disease pathogen is advantageous for eliciting an immune response in a mammal that has infectious diseases responsive to an immune response. An infectious disease responsive to an immune response is a disease caused by a pathogen in which the elicitation of an immune response against the pathogen can result in a prophylactic or therapeutic effect as previously described herein. Such a method provides a long term, targeted therapy for primary lesions (e.g., granulomas) resulting from the propagation of a pathogen. As used herein, the term “lesion” refers to a lesion formed by infection of a mammal with a pathogen. A therapeutic composition for use in the elicitation of an immune response in a mammal that has an infectious disease comprises a pathogen antigen-encoding recombinant molecule, alone or in combination with a cytokine-encoding recombinant molecule of the present invention, combined with a liposome delivery vehicle. Similar to the mechanism described above for the treatment of cancer, eliciting an immune response in a mammal that has an infectious disease with immunogens from the infectious disease pathogens with or without cytokines can result in increased T cell, natural killer cell, and macrophage cell activity that overcome the relative lack of immune response to a lesion formed by a pathogen. Preferably, expression of the immunogen in a tissue of a mammal that has an infectious disease produces a result which includes alleviation of the disease, regression of established lesions associated with the disease, alleviation of symptoms of the disease, immunization against the disease and stimulation of effector cell immunity against the disease.

A therapeutic composition of the present invention is particularly useful for eliciting an immune response in a mammal that has an infectious diseases caused by pathogens, including, but not limited to, bacteria (including intracellular bacteria which reside in host cells), viruses, parasites (including internal parasites), fungi (including pathogenic fungi) and endoparasites. Preferred infectious diseases to treat with a therapeutic composition of the present invention include chronic infectious diseases, and more preferably, pulmonary infectious diseases, such as tuberculosis. Particularly preferred infectious diseases to treat with a therapeutic composition of the present invention include human immunodeficiency virus (HIV), Mycobacterium tuberculosis, herpesvirus, papillomavirus and Candida.

In one embodiment, an infectious disease a therapeutic composition of the present invention is a viral disease, and preferably, is a viral disease caused by a virus which includes, human immunodeficiency virus, and feline immunodeficiency virus.

A therapeutic composition of the present invention which may include a nucleic acid molecule encoding an immunogen that is an allergen is advantageous for eliciting an immune response in a mammal that has a disease associated with allergic inflammation. A disease associated with allergic inflammation is a disease in which the elicitation of one type of immune response (e.g., a Th2-type immune response) against a sensitizing agent, such as an allergen, can result in the release of inflammatory mediators that recruit cells involved in inflammation in a mammal, the presence of which can lead to tissue damage and sometimes death. A therapeutic composition for use in the elicitation of an immune response in a mammal that has a disease associated with allergic inflammation comprises an allergen-encoding recombinant molecule, alone or in combination with a cytokine-encoding recombinant molecule, combined with a PRRL:liposome delivery vehicle.

Preferred diseases associated with allergic inflammation which are preferable to treat using the method and composition of the present invention include, allergic airway diseases, allergic rhinitis, allergic conjunctivitis and food allergy.

A therapeutic composition of the present invention is particularly useful for initiating, wound healing, osteogenesis, bone engraftment and/or angiogenesis and fibrosis formation. Angiogenesis and fibrosis formation are key components of the function of the growth and repair process of the human body. Insufficient angiogenic ability can increase the severity of cardiovascular disorders such as peripheral vascular disease (PVD). In PVD, the arteries that carry blood to the arms or legs become narrowed or clogged, slowing or stopping the flow of blood. The disease most often affects the legs. Many people live with the symptoms of PVD, such as pain or numbness in the legs or arms, because they believe it is a normal part of aging. Stimulation of angiogenesis has been explored as one way to alleviate the underlying cause of PVD.

Angiogenesis also plays a role in wound healing. The response to a wound is a very primitive, yet essential innate host immune response for restoration of the integrity of the injured tissue. Injury in higher vertebrates involves a rapid repair process that leads to a fibrotic scar. Healing of a wound from trauma, infection, or foreign bodies is mediated largely by cytokines that accentuate inflammation and healing. The initial insult triggers coagulation and an acute local inflammatory response followed by recruitment of mesenchymal cells and proliferation. Out of control inflammation due to excess Th1 response of the local area surrounding a wound can lead to non-healing wounds. Uncontrolled matrix accumulation leads to fibrotic sequelae and scarring. The balance between inflammation and matrix growth into the healing wound is modulated by the proper mix of cytokines in the local area.

Osteosarcoma is one of the most common primary bone tumor affecting human adolescents and young adults. Traditional therapy has required amputation followed by chemotherapy to prolong survival. Limb salvage with massive cortical allografts is a commonly used alternative to amputation. Multiple studies have shown that survival is not adversely affected with limb salvage treatment and for many patients there is a perceived improvement in the quality of life. Despite success in the surgical technique, profound post-operative complications frequently occur. These complications include osteomyelitis, non-union and fracture of the graft. Osteomyelitis is the most common complication associated with allograft limb salvage. Osteomyelitis is rarely cured and commonly results in multiple revision surgeries, chronic pain, poor use of the limb and, in some cases, amputation. The common theme in the three above examples is the modulation of the angiogenic and fibrotic response. With the complications associated with wound healing and bone engraftment there is a need for compounds which increase healing time and success of engraftment operations.

Preferably, the therapeutic composition of the present invention may be administered to to a mammal thereby eliciting angiogenesis in the mammal. The method includes the step of administering to the mammal a therapeutic composition by a route of administration selected from subcutaneous and intramuscular administration. The therapeutic composition includes: (a) a liposome delivery vehicle; and (b) pattern recognition receptor ligand. The pattern recognition receptor ligand may comprise at least one Toll-like receptor ligand, such as but not limited to, extracts of gram-positive bacteria, extracts of mycobacteria, extracts of yeast, lipopolysaccharides (LPS), peptidoglycans, lipopeptides, lipoteichoic acids, flagellin, bacterial DNA, double-stranded RNA, zymosan, and imidazoquinoline compounds. Wherein said liposome delivery vehicle comprises lipids, such as but not limited to, multilamellar vesicle lipids, cationic lipids, extruded lipids, a combination of a cationic lipid and a neutral lipid, and a combination of a cationic lipid and a sterol.

The therapeutic composition of the present invention may be released over an extended period of time by combining with inert matrixes, such as but not limited to, gelatin and collagen. DNA isostabilizing agent may also be added to the delivery vehicle, such as but not limited to the following alone or in combination: betaine, trimetylamine n-oxide, and L-carnitine. Furthermore the lipid delivery vehicle may contain a DNA condensing agent, such as but not limited to the following alone or in combination: poly (L-lysine), spermidine, or spermine.

The therapeutic composition may have a toll receptor ligand to lipid ratio of from about 1:1 to about 1:64 and the mammals to be treated may be of humans, dogs, cats, mice, rats, sheep, cattle, horses and pigs.

In an alternate embodiment, the therapeutic composition of the present invention may be administered via a sustained release polymer. Poly (L-lactide) (PLA) microspheres containing cationic liposomes complexed to Toll-like receptor ligand molecules and antigens (including either peptides, proteins, carbohydrates, glycolipids, lipoproteins or other antigens or combinations thereof) and formulated as microspheres, may be manufactured. Other polymers (e.g., poly (L-lactide-co-glycolides) capable of encapsulating the Liposome-Toll like receptor ligand-antigen complexes (LATLC) and providing slow but steady release in vitro in biological fluids would also be acceptable.

Liposome-Toll like receptor ligand-antigen complexes (LATLC) would be formulated with PLA in organic solvents, then extruded and condensed to form microspheres of PLA that have encapsulated the LATLC. The most desirable formulation will consist of PLA microspheres of 1 to 10 μm diameter. This diameter is most desirable because it is the size most readily taken up by antigen presenting cells such as dendritic cells and macrophages. The polymers will be designed to result in the sustained release of the LATLC in tissues over a period of 1 to 6 months. The microspheres with entrapped LATLC can be administered by a variety of routes, including SC, IM, intradermal, and a variety of mucosal routes including orally, intranasally, inhalationally, intrarectally, and transcutaneously.

In another embodiment of the present invention, the therapeutic composition of the present invention as described herein at therapeutically effective concentrations or dosages may be combined with a pharmaceutically or pharmacologically acceptable carrier, excipient or diluent, either biodegradable or non-biodegradable. As used herein, a pharmaceutically acceptable excipient refers to any substance suitable for delivering a therapeutic composition useful in the method of the present invention to a suitable in vivo site. Preferred pharmaceutically acceptable excipients are capable of maintaining a pattern recognition receptor ligand and/or nucleic acid molecule of the present invention in a form that, upon arrival of the PRRL and/or nucleic acid molecule to a cell, the PRRL and/or nucleic acid molecule is capable of entering the cell and being expressed by the cell if the nucleic acid molecule encodes a protein to be expressed. Suitable excipients of the present invention include excipients or formularies that transport, but do not specifically target a PRRL and/or nucleic acid molecule to a cell (also referred to herein as non-targeting carriers). Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium atuminum silicate, triethanolamine, polyvinyl alcohol, polyvinylpyrrolidone, sugars and starches. Exemplary examples of carriers include, but are by no means limited to, water, phosphate buffered saline, Ringer's solution, dextrose solution, serum-containing solutions, Hank's solution, other aqueous physiologically balanced solutions, oils, esters, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, mixtures thereof and the like. Aqueous carriers can contain suitable auxiliary substances required to approximate the physiological conditions of the recipient, for example, by enhancing chemical stability and isotonicity. Particularly preferred excipients include non-ionic diluents, with a preferred non-ionic buffer being 5% dextrose in water (DW5). See, for example, Remington: The Science and Practice of Pharmacy, 2000, Gennaro, A R ed., Eaton, Pa.: Mack Publishing Co.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol. Therapeutic compositions of the present invention can be sterilized by conventional methods and/or lyophilized.

The invention provides kits for carrying out the methods of the invention. Accordingly, a variety of kits are provided. The kits may be used for any one or more of the following (and, accordingly, may contain instructions for any one or more of the following uses): use for therapeutically or prophylactially treating an individual against a pathogenic organism such as a viral, fungal or bacterial infection; treating some forms of cancer in an individual; preventing the spread or metastasis of some forms of cancer; preventing one or more symptoms of some forms of cancer; reducing severity of one or more symptoms associated with cancer; delaying development of cancer in an individual; or vaccinating an individual against some forms of cancer.

The kits of the invention comprise one or more containers comprising the therapeutic composition of the present invention and a suitable excipient as described herein and a set of instructions, generally written instructions although electronic storage media (e.g., magnetic diskette or optical disk) containing instructions are also acceptable, relating to the use and dosage of the therapeutic composition of the present invention for the intended treatment. The instructions included with the kit generally include information as to dosage, dosing schedule, and route of administration for the intended treatment. The containers of the therapeutic composition of the present invention may be unit doses, bulk packages (e.g., multi-dose packages) or sub-unit doses.

The therapeutic composition of the present invention may be packaged in any convenient, appropriate packaging.

As will be appreciated by one knowledgeable in the art, the therapeutic composition of the present invention may be combined or used in combination with other treatments known in the art.

Another embodiment contemplates the incorporation of the composition of the present invention into a medical device that is then positioned to a desired target location within the body, whereupon the composition of the present invention elutes from the medical device. Thus, by way of example, the present invention will be described in relation to vascular stents. However, it should be understood that the following embodiments relate to any medical device incorporating the composition of the present invention, and is not limited to any particular type of medical device.

The devices of this invention provide a therapeutically effective amount of the composition of the present invention to a targeted site such as a diseased or injured bodily tissue or organ. The precise desired therapeutic effect will vary according to the condition to be treated, the formulation to be administered, and a variety of other factors that are appreciated by those of ordinary skill in the art. The amount of the composition of the present invention needed to practice the claimed invention also varies with the nature of the PRRL used.

In one embodiment, the medical device to be coated with the composition of the present invention is a stent or catheter for performing or facilitating a medical procedure. Accordingly, the present invention may be used in conjunction with any suitable or desired set of stent components and accessories, and it encompasses any of a multitude of stent designs. These stent designs may include for example a basic solid or tubular flexible stent member or a balloon catheter stent, up to complex devices including multiple tubes or multiple extruded lumens, as well as various accessories such as guidewires, probes, ultrasound, optic fiber, electrophysiology, blood pressure or chemical sampling components. In other words, the present invention may be used in conjunction with any suitable stent or catheter design, and is not limited to a particular type of catheter.

As used herein, “medical device” refers to a device that is introduced temporarily or permanently into a mammal for the prophylaxis or therapy of a medical condition. These devices include any that are introduced subcutaneously, percutaneously or surgically to rest within an organ, tissue or lumen. Medical devices may include stents, synthetic grafts, artificial heart valves, artificial hearts and fixtures to connect the prosthetic organ to the vascular circulation, venous valves, abdominal aortic aneurysm (AAA) grafts, inferior venal caval filters, catheters including permanent drug infusion catheters, embolic coils, embolic materials used in vascular embolization (e.g., PVA foams), mesh repair materials, a Dracon vascular particle orthopedic metallic plates, rods and screws and vascular sutures.

For purposes of this invention, “elution” refers to any process of release that involves extraction or release by direct contact of the coating with bodily fluids.

In one embodiment, the medical can be designed to have pores for the delivery of the composition of the present invention to the desired bodily location, and can be prepared by the method disclosed in U.S. Pat. No. 5,972,027, which is incorporated herein by reference. Briefly, the method comprises providing a powdered metal or polymeric material, subjecting the powder to high pressure to form a compact, sintering the compact to form a final porous metal or polymer, forming a stent from the porous metal and, optionally, loading at least the composition of the present invention (and optionally one or more additional drugs) into the pores. For example, the stent may be impregnated with the composition of the present invention and optionally one or more additional drugs by any known process in the art, including high pressure loading in which the stent is placed in a bath of the desired drug or drugs and subjected to high pressure or, alternatively, subjected to a vacuum. The drug(s) may be carried in a volatile or non-volatile solution. In the case of a volatile solution, following loading of the drug(s), the volatile carrier solution may be volatilized. In the case of the vacuum, the air in the pores of the metal stent is evacuated and replaced by the drug-containing solution. Alternatively, rather than loading the porous stent with the drug, the stent is instead implanted in the desired bodily location, and then the drug is injected through a delivery tubing to the hollow stent and then out the pores in the stent to the desired location.

In another embodiment, the stent can be designed to contain reservoirs or channels which could be loaded with the composition of the present invention as described in U.S. Pat. No. 6,273,913 B1, which is incorporated herein by reference. A coating or membrane of biocompatible material could be applied over the reservoirs which would control the diffusion of the drug from the reservoirs to the artery wall. One advantage of this system is that the properties of the coating can be optimized for achieving superior biocompatibility and adhesion properties, without the additional requirement of being ale to load and release the drug. The size shape, position, and number of reservoirs can be used to control the amount of drug, and therefore the dose delivered.

The stent can be made of virtually any biocompatible material having physical properties suitable for the design, and can be biodegradable or nonbiodegradable. The material can be either elastic or inelastic, depending upon the flexibility or elasticity of the polymer layers to be applied over it. Accordingly, the medical devices of this invention can be prepared in general from a variety of materials including ordinary metals, shape memory alloys, various plastics and polymers, carbons or carbon fibers, cellulose acetate, cellulose nitrate, silicone and the like.

For example, a medical device, such as but not limited to a stent, according to this invention can be composed of polymeric or metallic structural elements onto which a matrix is applied or the stent can be a composite of the matrix intermixed with a polymer.

Suitable biocompatible metals for fabricating the expandable stent include high grade stainless steel, titanium alloys including NiTi (a nickel-titanium based alloy referred to as Nitinol), cobalt alloys including cobalt-chromium-nickel alloys such as Elgiloy® and Phynox®, a Niobium-Titanium (NbTi) based alloy, tantalum, gold, and platinum-iridium.

Suitable nonmetallic biocompatible materials include, but are not limited to, polyamides, polyolefins (e.g., polypropylene, polyethylene etc.), nonabsorbable polyesters (i.e. polyethylene terephthalate), and bioabsorbable aliphatic polyesters (e.g., homopolymers and copolymers of lactic acid, glycolic acid, lactide, glycolide, para-dioxanone, trimethylene carbonate, ε-caprolactone, etc. and blends thereof).

Matrix

In one embodiment, the medical device such as a stent or graft is coated with a matrix. The matrix used to coat the stent or graft according to this invention may be prepared from a variety of materials. A primary requirement for the matrix is that it be sufficiently elastic and flexible to remain unruptured on the exposed surfaces of the stent or synthetic graft.

(A) Naturally Occurring Materials

The matrix may be selected from naturally occurring substances such as film-forming polymeric biomolecules that may be enzymatically degraded in the human body or are hydrolytically unstable in the human body such as fibrin, fibrinogen, heparin, collagen, elastin, and absorbable biocompatable polysaccharides such as chitosan, starch, fatty acids (and esters thereof), glucoso-glycans, hyaluronic acid, carbon, laminin, and cellulose.

(B) Synthetic Materials

In one embodiment, matrix that is used to coat the stent or synthetic graft may be selected from any biocompatible polymeric material capable of holding the composition of the present invention. The polymer chosen must be a polymer that is biocompatible and minimizes irritation to the vessel wall when the stent is implanted. The polymer may be either a biostable or a bioabsorbable polymer depending on the desired rate of release or the desired degree of polymer stability.

Suitable materials for preparing a polymer matrix include, but are not limited to, polycarboxylic acids, cellulosic polymers, silicone adhesive, fibrin, gelatin, polyvinylpyrrolidone, maleic anhydride polymers, polyamides, polyvinyl alcohols, polyethylene glycols, polyethylene oxides, glycosaminoglycans, polysaccharides, polyesters, poly(amino acids)polyurethanes, segmented polyurethane-urea/heparin, silicons, polyorthoesters, polyanhydrides, polycarbonates, polypropylenes, poly-L-lactic acids, polyglycolic acids, polycaprolactones, polyhydroxybutyrate valerates, polyacrylamides, polyethers, polyalkylenes oxalates, polyamides, poly(iminocarbonates), polyoxaesters, polyamidoesters, polyoxaesters containing amido groups, polyphosphazenes, vinyl halide polymers, polyvinylidene halides, polyacrylonitrile, polyvinyl ketones, polyvinyl aromatics (e.g., polystyrene), etheylene-methyl methacrylate copolymers, acrylonitrile-styrene copolymers, ABS resins and ethylene-vinyl acetate copolymers; polyamides,such as Nylon 66 and polycaprolactam; alkyl resins; polycarbonates; polyoxymethylenes; polyimides; polyethers; epoxy resins, polyurethanes; rayon; rayon-triacetate, cellulose, cellulose acetate, cellulose acetate butyrate; cellophane; cellulose nitrate; cellulose propionate; cellulose ethers (i.e. carboxymethyl cellulose and hydoxyalkyl celluloses) and mixtures and copolymers thereof.

The polymers used for coatings are preferably film-forming polymers that have molecular weight high enough as to not be waxy or tacky. The polymers also preferably adhere to the stent and are not so readily deformable after deposition on the stent as to be able to be displaced by hemodynamic stresses. The polymers molecular weight are preferably high enough to provide sufficient toughness so that the polymers will not be rubbed off during handling or deployment of the stent and will not crack during expansion of the stent.

In one embodiment, the matrix coating can include a blend of a first co-polymer having a first, high release rate and a second co-polymer having a second, lower release rate relative to the first release rate as described in U.S. Pat. No. 6,569,195 B2, which is incorporated herein by reference. The first and second copolymers are preferably erodible or biodegradable. In one embodiment, the first copolymer is more hydrophilic than the second copolymer. For example, the first copolymer can include a polylactic acid/polyethylene oxide (PLA-PEO) copolymer and the second copolymer can include a polylactic acid/polycaprolactone (PLA-PCL) copolymer. Formation of PLA-PEO and PLA-PCL copolymers is well known to those skilled in the art. The relative amounts and dosage rates of the composition of the present invention delivered over time can be controlled by controlling the relative amounts of the faster releasing polymers relative to the slower releasing polymers. For higher initial release rates the proportion of faster releasing polymer can be increased relative to the slower releasing polymer. If most of the dosage is desired to be released over a long time period, most of the polymer can be the slower releasing polymer.

Alternatively, a top coating can be applied to delay release of the pharmaceutical agent, or could be used as the matrix for the delivery of a different pharmaceutically active material. For example, layering of coatings of fast and slow hydrolyzing copolymers can be used to stage release of the drug or to control release of different agents placed in different layers. Polymers with different solubilities in solvents can be used to build up different polymer layers that may be used to deliver different drugs or control the release profile of a drug. For example since ε-caprolactone-co-lactide elastomers are soluble in ethyl acetate and ε-caprolactone-co-glycolide elastomers are not soluble in ethyl acetate. A first layer of ε-caprolactone-co-glycolide elastomer containing a drug can be over coated with ε-caprolactone-co-glycolide elastomer using a coating solution made with ethyl acetate as the solvent. As will be readily appreciated by those skilled in the art numerous layering approaches can be used to provide the desired drug delivery.

In one embodiment the coating is formulated by mixing the composition of the present invention and optionally one or more additional therapeutic agents with the coating polymers in a coating mixture. The composition of the present invention and the therapeutic agent may be present as a liquid, a finely divided solid, or any other appropriate physical form. Optionally, the mixture may include one or more additives, e.g., nontoxic auxiliary substances such as diluents, carriers, excipients, stabilizers or the like. Other suitable additives may be formulated with the polymer and the composition of the present invention and pharmaceutically active agent or compound. For example hydrophilic polymers selected from the previously described lists of biocompatible film forming polymers may be added to a biocompatible hydrophobic coating to modify the release profile (or a hydrophobic polymer may be added to a hydrophilic coating to modify the release profile). One example would be adding a hydrophilic polymer selected from the group consisting of polyethylene oxide, polyvinyl pyrrolidone, polyethylene glycol, carboxylmethyl cellulose, hydroxymethyl cellulose and combination thereof to an aliphatic polyester coating to modify the release profile. Appropriate relative amounts can be determined by monitoring the in vitro and/or in vivo release profiles for the composition of the present invention and the therapeutic agents.

Biodegradable Matrix

In one embodiment, the matrix is a synthetic or naturally occurring biodegradable polymer such as aliphatic and hydroxy polymers of lactic acid, glycolic acid, mixed polymers and blends, polyhydroxybutyrates and polyhydroxy-valeriates and corresponding blends, or polydioxanon, modified starch, gelatine, modified cellulose, caprolactaine polymers, polyacrylic acid, polymethacrylic acid or derivatives thereof, which will not alter the structure or function of the medical device. Such biodegradable polymers will disintegrate in a controlled manner (depending on the characteristics of the carrier material and the thickness of the layer(s) thereof), with consequent slow release of the composition of the present invention incorporated therein, while in contact with blood or other body fluids. A discussion of biodegradable coatings is provided in U.S. Pat. No. 5,788,979, which is specifically incorporated herein by reference.

Application of the Matrix to the Medical Device

In accordance with one embodiment of the present invention, the composition of the present invention is applied as an integral part of a coating on at least the exterior surface of the stent. The solution is applied to the stent and the solvent is allowed to evaporate, thereby leaving on the stent surface a coating of the polymer and the therapeutic substance. Typically, the solution can be applied to the stent by any suitable mean such as, for example, by immersion, spraying, or deposition by plasma or vapor deposition. In order to coat a medical device such as a stent, the stent is dipped or sprayed with a liquid solution of the matrix of moderate viscosity. After each layer is applied, the stent is dried before application of the next layer. In one embodiment, a thin, paint-like matrix coating does not exceed an overall thickness of 100 microns. Whether one chooses application by immersion or application by spraying depends principally on the viscosity and surface tension of the solution, however, it has been found that spraying in a fine spray such as that available from an airbrush will provide a coating with the greatest uniformity and will provide the greatest control over the amount of coating material to be applied to the stent. In either a coating applied by spraying or by immersion, multiple application steps are generally desirable to provide improved coating uniformity and improved control over the amount of therapeutic substance to be applied to the stent. The amount of the composition of the present invention to be included on the stent can be readily controlled by applying multiple thin coats of the solution while allowing it to dry between coats. The overall coating should be thin enough so that it will not significantly increase the profile of the stent for intravascular delivery by catheter. The adhesion of the coating and the rate at which the composition of the present invention is delivered can be controlled by the selection of an appropriate bioabsorbable or biostable polymer and by the ratio of composition of the present invention to polymer in the solution.

In order to provide the coated stent according to this embodiment, a solution which includes a solvent, a polymer dissolved in the solvent, the composition of the present invention dispersed in the solvent, and optionally a cross-linking agent, is first prepared. It is important to choose a solvent, and polymer that are mutually compatible with the composition of the present invention. It is essential that the solvent is capable of placing the polymer into solution at the concentration desired in the solution. It is also essential that the solvent and polymer chosen do not chemically alter the therapeutic character of the composition of the present invention. However, the composition of the present invention only needs to be dispersed throughout the solvent so that it may be either in a true solution with the solvent or dispersed in fine particles in the solvent. Preferable conditions for the coating application are when the polymer and composition of the present invention have a common solvent. This provides a wet coating that is a true solution. Less desirable, yet still usable are coatings that contain the composition of the present invention as a solid dispersion in a solution of the polymer in solvent. Under the dispersion conditions, care must be taken if a slotted or perforated stent is used to ensure that the particle size of the dispersed pharmaceutical powder, both the primary powder size and its aggregates and agglomerates, is small enough not to cause an irregular coating surface or to clog the slots or perferations of the stent. In cases where a dispersion is applied to the stent and it is desired to improve the smoothness of the coating surface or ensure that all particles of the drug are fully encapsulated in the polymer, or in cases where it is desirable to slow the release rate of the drug, deposited either from dispersion or solution, a clear (polymer only) top coat of the same polymer used to provide sustained release of the drug or another polymer can be applied that further restricts the diffusion of the drug out of the coating.

The composition coats the exterior and interior surfaces of the stent and, as it solidifies, encapsulates these surfaces in the polymer/composition of the present invention formulation. The dried stent thus includes a coating of the composition of the present invention on its surfaces. Preferably, the immersion methods are adapted such that the solution or suspension does not completely fill the interior of the stent or block the orifice. Methods are known in the art to prevent such an occurrence, including adapting the surface tension of the solvent used to prepare the composition, clearing the lumen after immersion, and placement of an inner member with a diameter smaller than the lumen in such a way that a passageway exists between all surfaces of the stent and the inner member. An alternative to dipping the distal end of the stent is to spray-coat the exterior and interior surfaces with a vaporized form of the composition comprising the composition of the present invention.

In one embodiment, the matrix is chosen such that it adheres tightly to the surface of the stent or synthetic graft. This can be accomplished, for example, by applying the matrix in successive thin layers. Each layer of matrix may incorporate the antibodies. Alternatively, composition of the present invention may be applied only to the layer in direct contact with the vessel lumen. Different types of matrices may be applied successively in succeeding layers.

The solvent is chosen such that there is the proper balance of viscosity, deposition level of the polymer, solubility of the pharmaceutical agent, wetting of the stent and evaporation rate of the solvent to properly coat the stents. In the preferred embodiment, the solvent is chosen such the composition of the present invention and the polymer are both soluble in the solvent. In some cases, the solvent must be chosen such that the coating polymer is soluble in the solvent and such that pharmaceutical agent is dispersed in the polymer solution in the solvent. In that case the solvent chosen must be able to suspend small particles of the composition of the present invention without causing them to aggregate or agglomerate into collections of particles that would clog the slots of the stent when applied. Although the goal is to dry the solvent completely from the coating during processing, it is a great advantage for the solvent to be non-toxic, non-carcinogenic and environmentally benign. Mixed solvent systems can also be used to control viscosity and evaporation rates. In all cases, the solvent must not react with or inactivate the composition of the present invention or react with the coating polymer. Preferred solvents include, but are not limited to, acetone, N-methylpyrrolidone (NMP), dimethyl sulfoxide (DMSO), toluene, xylene, methylene chloride, chloroform, 1,1,2-trichloroethane (TCE), various freons, dioxane, ethyl acetate, tetrahydrofuran (THF), dimethylformamide (DMF), dimethylacetamide (DMAC), water, and buffered saline.

In one embodiment, a stent is coated with a mixture of a pre-polymer, cross-linking agents and the composition of the present invention, and then subjected to a curing step in which the pre-polymer and cross-linking agents cooperate to produce a cured polymer matrix containing the composition of the present invention. The curing process involves evaporation of the solvent and the curing and cross-linking of the polymer. Certain silicone materials can be cured at relatively low temperatures, (i.e., room temperature to 50° C.) in what is known as a room temperature vulcanization (RTV) process. Of course, the time and temperature may vary with particular silicones, cross-linkers and biologically active species.

Generally, the amount of coating to be placed on the catheter will vary with the polymer, and may range from about 0.1 to 40 percent of the total weight of the catheter after coating. The polymer coatings may be applied in one or more coating steps depending on the amount of polymer to be applied.

Addition of Composition of the Present Invention to the Matrix

The composition of the present invention can be incorporated into the matrix, either covalently or noncovalently, wherein the coating layer provides for the controlled release of the composition of the present invention from the coating layer. The composition of the present invention may be incorporated into each layer of matrix by mixing the composition of the present invention with the matrix coating solution. Alternatively, the composition of the present invention may be covalently or noncovalently coated onto the last layer of matrix that is applied to the medical device. The desired release rate profile of the composition of the present invention from the device can be tailored by varying the coating thickness, the radial distribution (layer to layer) of the composition of the present invention, the mixing method, the amount of the composition of the present invention, the combination of different matrix polymer materials at different layers, and the crosslink density of the polymeric material, as discussed below.

In one embodiment, the composition of the present invention is added to a solution containing the matrix. For example, the composition of the present invention can be incubated with a solution containing a polymer at an appropriate concentration of the composition of the present invention. It will be appreciated that the concentration of the composition of the present invention will vary and that one of ordinary skill in the art could determine the optimal concentration without undue experimentation. The composition of the present invention/polymer mixture is then applied to the device by any of the methods described herein.

The ratio of the composition of the present invention to polymer in the solution will depend on the efficacy of the polymer in securing the composition of the present invention onto the stent and the rate at which the coating is to release the composition of the present invention to the tissue of the blood vessel. More polymer may be needed if it has relatively poor efficacy in retaining the composition of the present invention on the stent and more polymer may be needed in order to provide an elution matrix that limits the elution of a very soluble composition of the present invention. A wide ratio of composition of the present invention to polymer could therefore be appropriate and could range from about 10:1 to about 1:100.

Deposition of the Composition of the Present Invention onto a Coated Stent

In another embodiment, a medical device of this invention such as a stent comprises a at least one layer of the composition of the present invention deposited on at least a portion of a coating layer of the stent. If desired, a porous layer can be deposited over the composition of the present invention layer, wherein the porous layer includes a polymer and provides for the controlled release of the composition of the present invention therethrough and further avoids degradation of the composition of the present invention. Methods of coating a stent according to this embodiment is disclosed in U.S. Pat. No. 6,299,604, which is specifically incorporated herein by reference.

In yet another embodiment, the composition of the present invention is covalently coupled to the matrix. In one embodiment, the composition of the present invention can be covalently coupled to the matrix through the use of hetero- or homobifunctional linker molecules. The use of linker molecules in connection with the present invention typically involves covalently coupling the linker molecules to the matrix after it is adhered to the stent. After covalent coupling to the matrix, the linker molecules provide the matrix with a number of functionally active groups that can be used to covalently couple one or more types of composition of the present invention. The linker molecules may be coupled to the matrix directly (i.e., through the carboxyl groups), or through well-known coupling chemistries, such as, esterification, amidation, and acylation. For example, the linker molecule could be a polyamine functional polymer such as polyethyleneimine (PEI), polyallylamine (PALLA) or polyethyleneglycol (PEG). A variety of PEG derivatives, e.g., mPEG-succinimidyl propionate or mPEG-N-hydroxysuccinimide, together with protocols for covalent coupling, are commercially available from Shearwater Corporation, Birmingham, Ala. (See also, Weiner, et al., J. Biochem. Biophys. Methods, 45:211-219 (2000), incorporated herein by reference). It will be appreciated that the selection of the particular coupling agent may depend on the type of delivery vehicle used in the composition of the present invention and that such selection may be made without undue experimentation.

Coating a Stent with the Composition of the Present Invention

In yet another embodiment, a thin layer of the composition of the present invention is covalently or noncovalently bonded to the exterior surfaces of the stent. In this embodiment, the stent surface is prepared to molecularly receive the composition of the present invention according to methods known in the art. If desired, a porous layer can be deposited over the composition of the present invention layer, wherein the porous layer includes a polymer and provides for the controlled release of the composition of the present invention therethrough and further avoids degradation of the composition of the present invention.

Compounded Medical Devices

In an alternative embodiment of a medical device according to the invention, the composition of the present invention is provided throughout the body of the medical device by mixing and compounding the composition of the present invention directly into the medical device polymer melt before forming the medical device. For example, the composition of the present invention can be compounded into materials such as silicone rubber or urethane. The compounded material is then processed by conventional method such as extrusion, transfer molding or casting to form a particular configuration. The medical device resulting from this process benefits by having the composition of the present invention dispersed throughout the entire medical device body. Thus, the composition of the present invention is present at the outer surface of the medical device when the medical device is in contact with bodily tissues, organs or fluids and acts to modulate an immune response.

The invention is further illustrated by the following non-limited examples. All scientific and technical terms have the meanings as understood by one with ordinary skill in the art. The specific examples which follow illustrate the methods in which the compositions of the present invention may be prepared and are not to be construed as limiting the invention in sphere or scope. The methods may be adapted to variation in order to produce compositions embraced by this invention but not specifically disclosed. Further, variations of the methods to produce the same compositions in somewhat different fashion will be evident to one skilled in the art.

### EXAMPLES

The examples herein are meant to exemplify the various aspects of carrying out the invention and are not intended to limit the invention in any way.

### Example 1

### Liposomes Markedly Enhance Activation of Innate Immunity and IFN-γ Release After Activation by Pattern Recognition Receptor Ligands (PRRL)

The ability of cationic liposomes to augment immune activation elicited by PRRL was assessed in vitro, using a spleen cell assay. Spleen cells Were prepared from normal ICR mice and added at a concentration of 5×10⁶ /ml in individual wells of 24-well plates. A series of different PRRL, including plasmid DNA (“DNA”), CpG oligonucleotides (“CpG”), an imidazoquinoline (R-848; InVivogen), and purified E coli endotoxin (“LPS”) were mixed With a cationic liposome to form complexes. The cationic liposome was prepared using equimolar amounts of DOTIM (octadecenoyloxy-ethyl-2-heptadecenyl-3-hydroxyethyl) and cholesterol and rehydration in a solution of 5% dextrose in water. To form specific PRRL-lipo-some complexes, 30 μmol of cationic liposome was added to 30 μl 5% dextrose in water, followed by addition of 3 μg of each PRLL and mixing by pipetting. To compare immune-stimulatory activity, 2.5 μl of the liposome-PRRL complex was added to each well of spleen cells, to achieve a final PRRL concentration of 500 ng/ml. To other wells, either the PRRL alone (50 ng/ml) or liposome alone was added. Cell cultures were incubated for 18 hours, then supernatants were collected and assayed for their concentration of IFN-γ, using a commercial EISA assay (R & D Systems). The IFN-γ concentrations were then plotted, shown in FIG. 1. These results demonstrate that liposome-PRRL complexes are much more potent activators of immune response and IFN-y release than the PRRL alone. Moreover, these results illustrate the general principal that the liposomes (and potentially other delivery systems as well) are capable of substantially altering the immune-stimulatory properties of diverse PRRL.

### Example 2

### Liposomes Alter Release of IL-10 After Activation by Pattern Recognition Receptor Ligands (PRRL)

The ability of liposomes to enhance release of a key immunosuppressive cytokine of the innate immune system was assessed using the spleen cell assay described above in Example 1. PRRL, with or without liposomes, were added at a final concentration of 500 ng/ml for 18 hours. The release of IL-10 into the supernatants was then assessed using an ELISA assay. Surprisingly, the data shown, in FIG. 2, illustrate that combining liposomes with PRRL can significantly alter the immunological properties of PRRL, by either augmenting release of IL-10 (eg, with DNA or R-848 as PRRL) or inhibiting IL-10 release (eg, CpG or LPS as PRRL). For example, liposomal-LPS strongly inhibited release of IL-10, compared to LPS alone, as did liposomal-CpG, whereas liposomal-R848 actually increased IL-10 release. Thus, formation of liposome-TLR ligand complexes alters the release of cytokines elicited by the ligand itself, in a TLR-ligand specific fashion. The alteration in cytokine release includes both stimulatory and inhibitory effects. In vivo, this alteration in cytokine release is likely to have important consequences for generation of T cell and B cell responses. These data provide additional proof that the immunologic properties of PRRL can be substantially modified by the addition of liposomes or other carrier molecules.

### Example 3

### Liposomes Enhance Release of TNF-α After Activation by Pattern Recognition Receptor Ligands (PRRL)

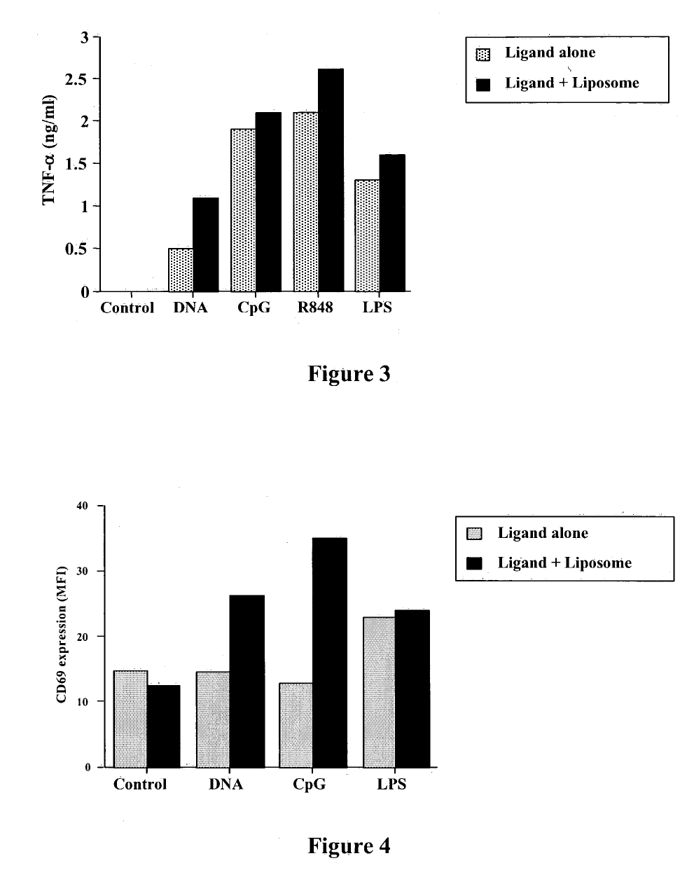

The ability of liposomes to enhance release of a second key stimulatory cytokine of the innate immune system was assessed using the spleen cell assay described above in Example 1. PRRL, with or without liposomes, were added at a final concentration of 500 ng/ml for 18 hours. The release of TNF-α into the supernatants was then assessed using an ELISA assay. The data shown, in FIG. 3, illustrate the liposome-complexed PRRL were more potent immune stimulators than the PRRL alone and provide further support for the principal of modification of PRRL properties by liposomes.

### Example 4

### Liposomes Alter the Regulation of Dendritic Cell Activation Following Exposure to Pattern Recognition Receptor Ligands (PRRL) in vitro

Spleen cells were incubated for 18 hours with PRRL, with or without liposomes, at a final PRRL concentration of 500 ng/ml. The spleen cells were then harvested and immunostained for surface expression of cell phenotypic markers (eg, macrophage and dendritic cell markers) as well as expression of the early activation marker CD69. The cells were then analyzed by flow cytometry and expression of CC69 on dendritic cells determined. The cell surface expression of CD69 was expressed as mean fluorescence intensity (MFI). In these experiments, it was found that liposome-complexed PRRL (in the case of plasmid DNA and CpG oligonucleotides) augmented cell activation, as reflected by upregulation of CD69 expression, see FIG. 4. Thus, liposomes can also serve to modify the cell activating properties of PRRL, as reflected by additional upregulation of CD69 expression.

### Example 5

### Peptides or Protein Antigens Complexed to Lipid-DNA Complexes

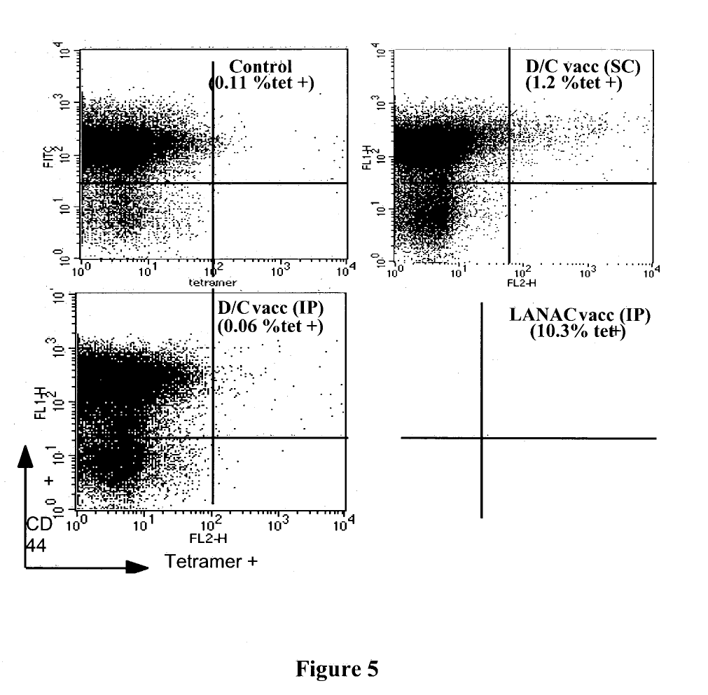

Experiments were performed using either peptides or protein antigens complexed to lipid-DNA complexes in order to assess antigen-specific responses directly. MHC-peptide tetramers were used in these experiments to quantitate CTL numbers and distribution. For tracking CTL responses to immunization with ova and the dominant CTL epitope (SIINFEKL; ova8) in C57B16 mice, Kb-ova8 tetramers were used. Kb-ova8 tetramers were provided by Ross Kedl, National Jewish Medical and Research Center, Denver, Colo. In these experiments, relatively low doses of peptides or proteins (typically 1 to 5 mg per immunization per mouse) were used in order to assess the efficiency of immunization. Surprisingly, it was discovered that liposome-antigen-nucleic acid complexes (LANAC) formulated with non-coding plasmid DNA and the ova8 peptide were extremely effective in eliciting CTL responses (FIG. 5, bottom right panel). To analyze the efficacy of LANAC immunization, mice were also immunized with autologous bone-marrow derived dendritic cells (DC) pulsed with ova8 peptide and CTL responses were also assessed by tetramers. These studies revealed the strong potency of LANAC vaccines for immunization against peptide antigens, as compared to dendritic cell vaccination.

CD8⁺ T cell responses MHC class I tetramers (H-2 Kb) and a model antigen (ovalbumin) were used to quantitate CD8⁺ T cell responses to immunization with peptide antigens (FIG. 5, top left panel). C57B16 mice (3-4 per group) were each immunized twice, one week apart with 1×10⁶ autologous bone-marrow derived dendritic cells (DC) that had been activated in vitro with LPS, then pulsed with 1 mM of the Kb-binding ova8 peptide (SIINFEKL). Mice were immunized with DC by either the SC route (FIG. 5, top right panel) or IP route (FIG. 5, bottom left panel). Another group of mice (FIG. 5, bottom right panel) was immunized with 5 mg of the ova8 peptide in LANAC, then injected IP. Five days after the second immunization, spleen cells were collected and immediately stained with Kb-ova8 tetramers (PE-labeled), with no in vitro restimulation, then with CD8-APC, CD44-FITC, and MHC class II-PE/Cy5 antibodies. Total CD8⁺ (after excluding MHC class II+ cells) were gated and analyzed for tetramer and CD44 staining; CD44 hi T cells represent the memory CTL staining. The number of tetramer+ cells was expressed as a percentage of total CD8+ cells analyzed. Immunization with ova8 peptide in LANAC induced a much stronger CTL response (FIG. 3, bottom right panel) than immunization with ova8-pulsed DC (10% of total spleen CD8 T cells were Ag specific, compared to 1-2% after DC vaccination). The IP route of immunization with LANAC was the most effective route for inducing T cell responses compared to SC or IM routes (data not shown here).

### Example 6

### LANAC and “Cross-Priming”

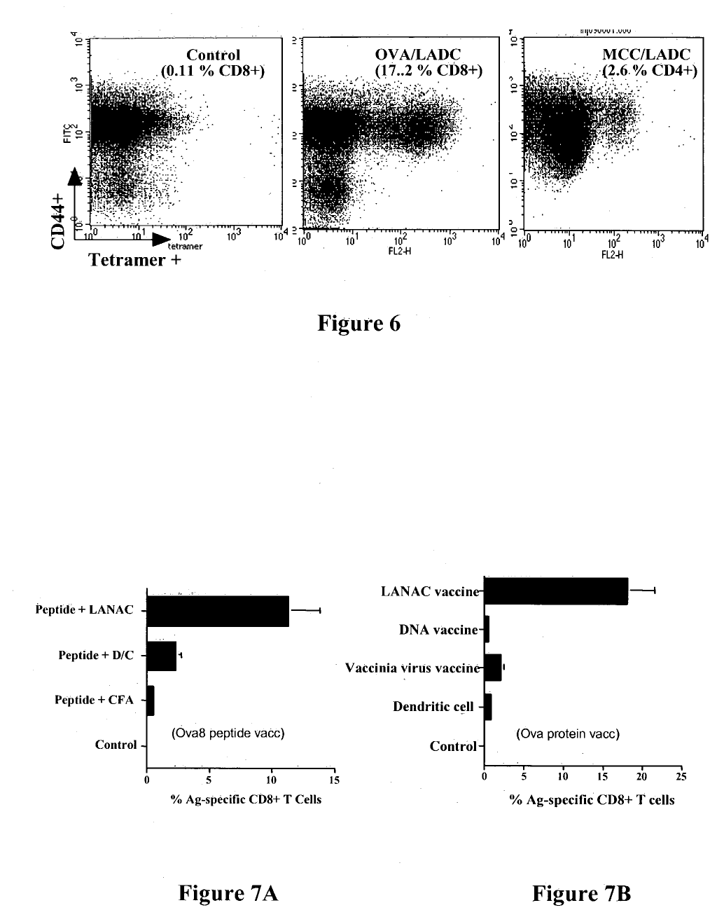

An experiment was performed to test whether LANAC could be used to efficiently “cross-prime” CTL responses against protein antigens. For these experiments, intact ova protein (which had been carefully filtered to remove any small MW peptides) was added to LANAC in lieu of peptides and mice were immunized as described in Example 5. Five days after the second immunization, CTL responses in the spleen were assessed using Kb-ova8 tetramers. Unexpectedly, LANAC were quite efficient in cross-priming CTL responses against protein antigens (FIG. 6, middle panel). The CTL responses to protein antigens elicited by LANAC were consistently 1.5 times stronger than responses to equivalent amounts (by weight) of peptide antigens. These results are very important, because the ability to elicit CTL responses against protein antigens makes it possible to eventually immunize humans with intact protein antigens without regard to MHC background or target antigen peptide specificity. Moreover, the responses in this system are elicited by a non-replicating system, whereas the best CTL responses previously demonstrated were elicited by replicating vectors such as viruses (vaccinia, adenovirus) or recombinant bacteria (Salmonella, Listeria). MHC class II tetramers were used to demonstrate that the LANAC system is capable of eliciting strong CD4 T cell responses. Mice immunized with the MCC antigen generated strong CD4 responses (FIG. 6, right panel) that exceeded those elicited by DC immunization (data not shown here). Thus, LANAC formulated vaccines were capable of eliciting strong and balanced T cell responses to protein antigens.

### Example 7

### Efficacy of LANAC Vaccines in Eliciting CTL Responses Compared to other Conventional Vaccines

The magnitude of Ag-specific CTL responses following immunization with peptides (FIG. 7A) or proteins (FIG. 7B) was assessed using Kb-ova8 tetramers. Immune responses to peptides were elicited by 50 mg peptide in complete Freund's adjuvant or peptide (1 mm)-pulsed DC. For protein vaccination mice were immunized with vaccinia virus encoding Ova by IV injection, by 100 mg plasmid DNA vector encoding ova by bilateral IM injection, or by immunization with DC pulsed with ova protein. Mice were immunized with LANAC containing 5 mg peptide or protein per mouse, then spleen cell tetramer responses were analyzed as described in Example 6. These data indicate that LANAC vaccines were consistently superior to other conventional types of vaccines for eliciting CTL responses.

The efficacy of LANAC vaccines in eliciting CTL responses was compared to other conventional vaccines, including peptide delivery systems (peptide-pulsed DC, peptide in complete Freund's adjuvant) and protein vaccines (ova-DNA vaccine, ova-vaccinia, ova-pulsed DC). C57B16 mice (4 animals per group) were immunized and CTL responses in the spleen were evaluated. In each case, particularly in the case of protein vaccines, LANAC formulated vaccines were clearly superior (FIG. 7A).

### Example 8

### Liposomes Enhance the Ability of PRRL to Serve as Vaccine Adjuvants for Elicitation of CTL Responses

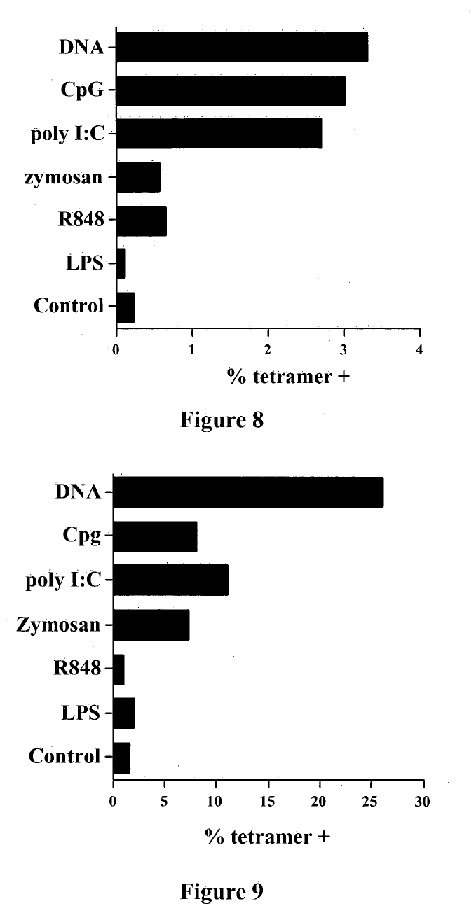

Experiments were conducted in mice to determine whether the addition of liposomes to different PRRL could augment the ability of PRRL to act as vaccine adjuvants. The PRRL evaluated included plasmid DNA, CpG oligonucleotides, polyI:C (synthetic mimic of ds-RNA), zymosan (from yeast cell wall), and R-848 and LPS. The immune response to a the model antigen ovalbumin was assessed in C57B16 mice, using MHC-peptide tetramer reagents to quantitate ovalbumin-specific CD8⁺ T cell (CTL) responses in vivo. Mice were immunized with 5 μg of ovalbumin administered along with the different liposome-PRRL complex vaccines twice, one week apart, then spleen and lung cells were analyzed by MHC-peptide tetramers and flow cytometry. To prepare the different vaccines, liposomes were first added to 1 ml 5% dextrose in water, followed by addition of 100 μg of each specific PRRL, followed by addition of ovalbumin protein. Mice were immunized with 200 μl of the liposome-PRRL complexes by the IP route. Spleen cells were then collected and immunostained first with MHC-peptide tetramer, followed by CD8 and CD44. Cells were then analyzed by flow cytometer and the mean percentage of antigen-specific CTL were calculated, based on group sizes of 3 mice per treatment group. Control mice were not vaccinated. The results of these experiments, shown in FIG. 8, indicate that liposomes complexes with the PRRL DNA, CpG oligos, poly I:C, zymosan and R-848 can all function as effective vaccine adjuvants for eliciting CTL responses in vivo. In contrast, liposomal-LPS was not an effective vaccine adjuvant. It was also found that DNA or CpG oligos administered alone With ovalbumin, or liposomes only plus ovalbumin, were not effective in eliciting CTL responses (data not shown). Thus, addition of a carrier molecule such as a liposome to PRRL can markedly enhance their effectiveness as vaccine adjuvants, particularly for eliciting CTL responses.

### Example 9

### Liposome-PRRL Complexes also Act as Effective Vaccine Adjuvants for Eliciting CTL Responses in the Lungs

Experiments were conducted, as described above in Example 8, to determine whether the liposome-PRRL complexes could also elicit strong CTL responses in lung tissues. Such a T cell response would be particularly desirable for immunization against inhaled pathogens such as influenza. Lung cells were collected after the second immunization with ovalbumin and analyzed by MHC-peptide tetramers for quantitation of ovalbumin-specific CTL responses. It Was found that similar patterns of CTL responses to immunization with liposome-PRRL vaccine adjuvants in the lungs as in the spleens of immunized mice (shown in FIG. 8), except that in the lungs zymosan was more effective and R-848 was ineffective, as compared to spleen responses, see FIG. 9. These data therefore indicate that liposome-PRRL vaccine adjuvants are also effective in eliciting T cell responses in peripheral tissues such as the lung, in addition to responses in lymphoid tissues such as the spleen.

### Example 10

### Determination of Whether 3-Part Liposome-Antigen-Nucleic Acid Complex is Required for Efficient Immunization

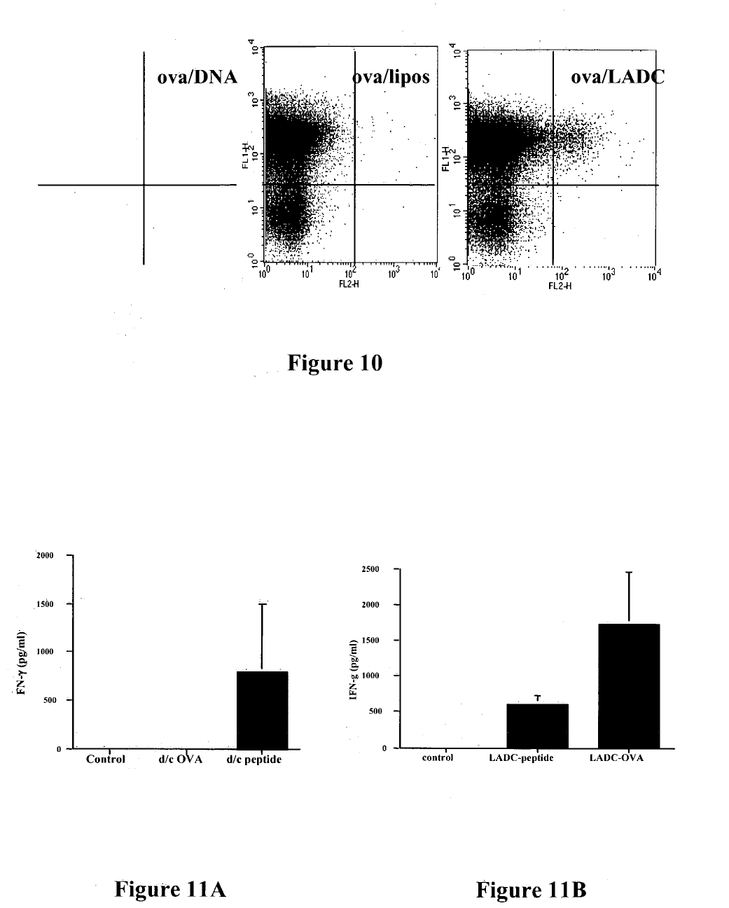

To determine whether the 3-part liposome-antigen-nucleic acid complex is required for efficient immunization, mice were immunized IP with 5 μg ova protein combined with various combinations of liposomes and DNA. Ova protein was added to equivalent amounts of plasmid DNA alone FIG. 10, first panel), liposomes alone (FIG. 10, second panel), or liposomes plus DNA (FIG. 10, third panel) (“ova/LADC”; note that “LADC” refers to the same formulation as “LANAC”). Spleen cells were collected and stained with Kb-ova8 tetramer to quantitate CTL responses. A very weak CTL response to DNA or liposome immunization alone was observed, compared to the response elicited by lipid-DNA complexes, indicating that in fact the 3-part combination of liposome, TLR ligand, and antigen is required for effective immunization.

### Example 11

### The Functional Capabilities of T Cells Elicited by Immunization with Liposome-Nucleic Acid Complexes

Experiments were also performed to assess the functional capabilities of T cells elicited by immunization with liposome-nucleic acid complexes. Spleen cells from mice, 4 per group, immunized with either ova8 peptide in LANAC (shown in FIG. 11A) or with peptide-pulsed DC (Shown in FIG. 11B) were restimulated in vitro with ova8 peptide and production of IFN-γ was assessed by ELISA. High levels of IFN-γ release were generated by T cells from mice immunized with ova8 peptide in LANAC or DC vaccines, whereas only ova protein formulated with LANAC (and not DC pulsed with ova) elicited IFN-γ release from CTL. LANAC immunization with other antigens (including Sendai virus, killed RSV, RSV M2 peptide, melanoma trp-2 antigen, and KLH protein) have also elicited production of high levels of IFN-γ by cultured T cells exposed to antigen in vitro (data not shown here). In addition, T cells from LANAC immunized mice (ova8 or trp2 peptides) also developed high levels of specific CTL activity after 5 days re-stimulation in vitro (data not shown here). These data demonstrate that LANAC immunization can in fact elicit functional Th1 and Tc1 T cell cytokine responses to a variety of different antigens.

### Example 15

### Assessing and Comparing the Efficiency of Distribution of LANAC to Lymphoid Organs

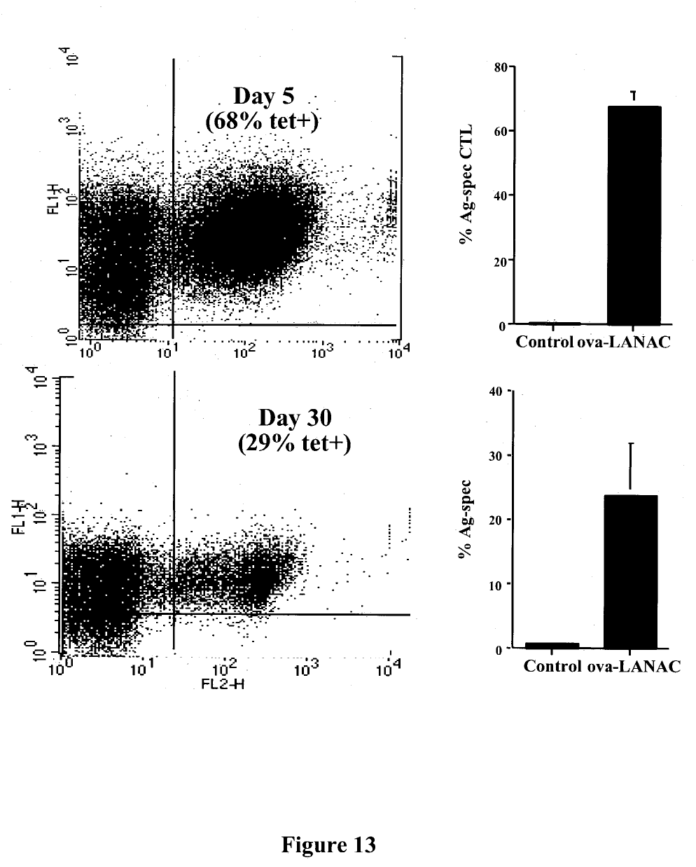

Experiments were performed to assess and compare the efficiency of distribution of LANAC to lymphoid organs, using BODIPY-labeled liposomes. Whether labeled complexes could be identified in lymph nodes either 6 hours or 24 hours after immunization with LANAC by either the SC or IP routes was determined. After SC immunization in the flank bilaterally, inguinal lymph nodes were harvested, whereas mesenteric lymph nodes were harvested after IP immunization. Lymph node cells were stained with antibodies for CD11b, CD11C, and MHC class II and analyzed by flow cytometry. Analysis gates were set on live cells so that only cell-associated LANAC were analyzed. Complexes were found to be present in lymph node from both sites of immunization, but that the distribution to draining lymph node was much more efficient after IP immunization (FIG. 15). The complexes were primarily contained within CD11b h1, CD11c lo, and class II intermediate cells. Labeled complexes associated with this same cell population in the peritoneal fluid after IP immunization were observed. Thus, it appears that uptake of LANAC in lymph node is much more efficient after IP injection and that this in turn correlates with the much stronger T cell responses elicited after IP injection than after SC or iv (data not shown here). The cell that contains labeled LANAC and has migrated to the mesenteric lymph node in FIG. 15 has a phenotype that is most consistent with a macrophage. However, several publications have now documented the presence of dendritic cells in the peritoneal cavity. These cells under resting conditions generally have a macrophage-like morphology, but can be induced to differentiate into classical dendritic cells by inflammatory stimuli or a mixture of different cytokines, particularly GM-CSF±TNF-α. Thus, it may well be the case that both true macrophages as well as macrophage-like dendritic cell precursors in the peritoneum endocytose LANAC after injection. Once these pre-DCs take up the complexes, they receive activating signals via TLRs, mature into more classical DC, and then migrate to regional lymph nodes where antigen presentation occurs. In the skin, it may be the case that classical DC such as Langerhans cells are more important f for LANAC uptake and antigen presentation. found that was equivalent in magnitude to that observed after LCMV infection (FIG. 13). Remarkably, in the lungs of these mice, 2 of every 3 CD8+ T cells were ova-specific. Large numbers of ova-specific CTL were also observed in the liver (data not shown here). Perhaps equally important, these CTL in peripheral tissues were found to also be long-lived. When mice were examined 30 days after the second immunization, nearly 30% of the total lung CD8⁺ T cells were still ova-specific; high numbers were also still present at 60 days (not shown). In addition, when spleen cells from day 30 mice were restimulated with ova peptide in vitro, they still produced high levels of IFN-γ (data not shown here). Thus, immunization with LANAC leads to the generation of extremely large numbers of memory CTL that reside for long periods of time in the lungs and other tissues. The presence of large numbers of long-lived memory T cells in the lungs is an ideal situation for mounting rapid responses to inhaled pathogens such as Yersinia.

### Example 14

### Evaluation of the Ability of Mucosally-Administered LANAC to Elicit Local and Systemic Immunity

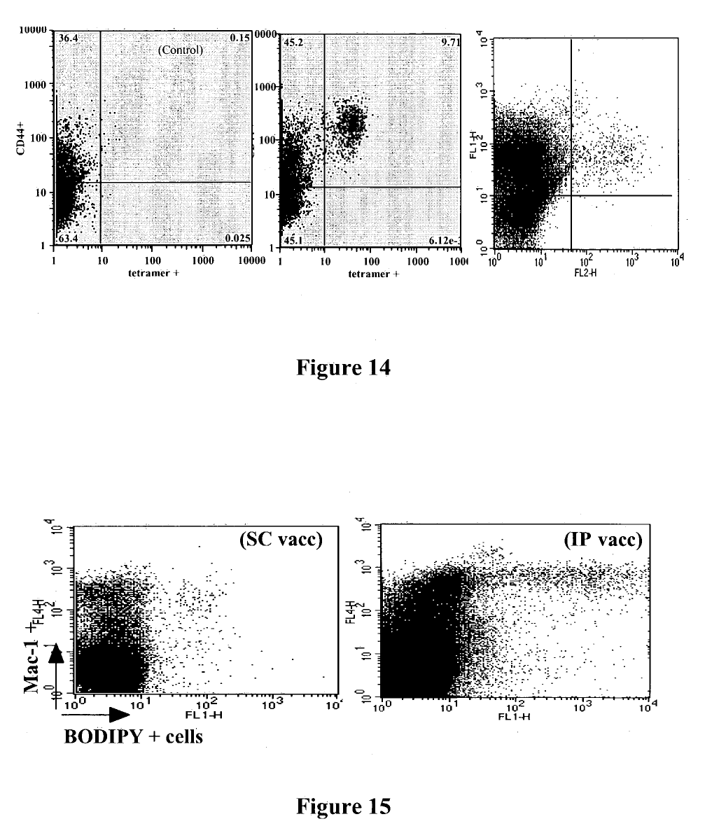

Since parenteral immunization using LANAC was so effective, the ability of mucosally-administered LANAC to elicit local and systemic immunity was evaluated. Mice were immunized twice with 5 mg/mouse of ova-LANAC by the oral (FIG. 14, center panel) or intranasal routes (FIG. 14, far right panel, then Ag-specific CTL responses were quantitated by tetramers. Lung cells were collected by enzymatic digestion prior to flow cytometry and analysis gates were set using live spleen lymphocytes.

Intransal immunization elicited a reasonably strong CTL response in the lungs, as detected by tetramers (FIG. 14, right panel). Most surprising however was the fact that oral administration of 5 mg ova protein was very effective in eliciting systemic CTL responses, including in the blood, spleen, liver, and lungs (FIG. 14, center panel). In fact, oral immunization was as effective as SC or IM immunization in eliciting CTL responses. Equally important, the CTL elicited by oral immunization were long-lived (at least 60 days) and functional, as evidenced by production of high levels of IFN-γ after ex vivo restimulation (data not shown here). Thus, the oral route of immunization with a protein vaccine using liposome-TLR ligand complexes as adjuvants may offer a means a rapid means of mucosal vaccination.

### Example 16

### Preparation of Cationic Lipid DNA Complexes (CLDC)

The cationic liposomes used in the following experiments (unless otherwise indicated) consisted of DOTAP (1,2 dioleoyl-3-trimethylammonium-propane) and cholesterol mixed in a 1:1 molar ratio, dried down in round bottom tubes, then rehydrated in 5% dextrose solution (D5W) by heating at 50° C. for 6 hours, as described previously (Solodin, et al, Biochemistry, 34:13537-13544 (1995), incorporated herein by reference in its entirety). Other lipids (e.g., DOTMA) were prepared similarly for some experiments as indicated. This procedure results in the formation of liposomes that consists of multilamellar vesicles (MLV), which the present inventors have found give optimal transfection efficiency as compared to small unilamellar vesicles (SUV). The production of MLVs and related “extruded lipids” is also described in Liu, et al., Nature Biotech., 15:167-173 (1997); and Templeton, et al., Nature Biotech., 15:647-652 (1997); both of which are incorporated herein by reference in their entirety. Plasmid DNA (pCR3.1, Invitrogen) was purified from E. coli as described previously, using modified alkaline lysis and polyethylene glycol precipitation (Liu, et al., 1997, supra). DNA for injection was resuspended in distilled water. Eukaryotic DNA (salmon testis and calf thymus) was purchased from Sigma Chemical Company. For many of the experiments reported here, the plasmid DNA did not contain a gene insert (unless otherwise noted), and is thus referred to as “non-coding” or “empty vector” DNA.

The cationic lipid TLR-ligand used in the experiments were prepared by gently adding TLR-ligand to a solution of lipid in 5% dextrose solution (D5W) at room temperature, then gently pipetting up and down several times to assure proper mixing. The TLR-ligand:lipid ratio was 1:8 (1.0 ug DNA to 8 nmol lipid). The complexes were used within 30-60 minutes of preparation, To prepare small unilamellar vesicles (SUV) used in some experiments (as indicated), the CLDC that were formed using MLV liposomes as described above were subjected to sonication for 5 minutes, as described previously (Liu et al., 1997, supra).

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow. The words “comprise,” “comprising,” “include,” “including,” and “includes” when used in this specification and in the following claims are intended to specify the presence of stated

### Example 12

### The Ability of Liposome-Nucleic Acid Vaccination to Elicit Humoral Immunity

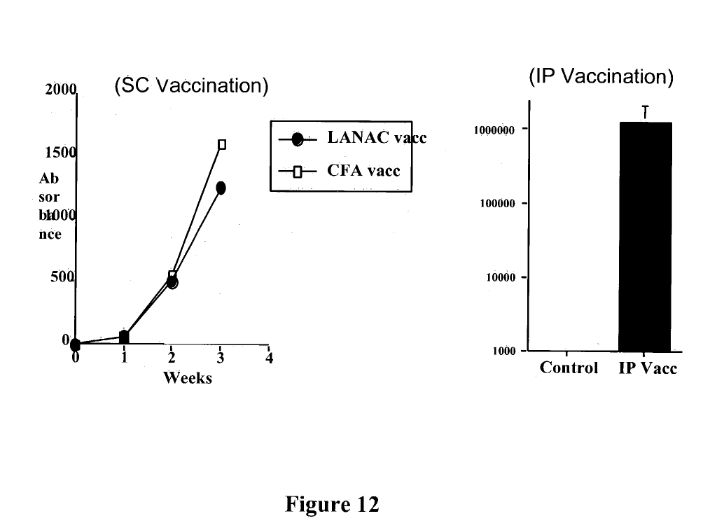

The ability of liposome-nucleic acid vaccination to elicit humoral immunity was also assessed, using the ova-LANAC system, but in BALB/c mice. BALB/c mice (4/group) were immunized twice by the SC route, two weeks apart, with 10 μg ova (protein) in either LANAC or complete Freund's adjuvant (CPA), see FIG. 12A and serial serum samples collected for determination of anti-ova titers by ELISA (FIG. 12B). SC immunization with LANAC elicited antibody responses nearly equivalent to those elicited by CFA. Mice immunized by the IP route developed much higher titers, with an average titer of 1:1.3 million. Similar titers have been observed after animals have been immunized with other antigens, including KLH (data not shown here). Thus, liposome-nucleic acid complexes are also very effective for efficient induction of humoral immunity. These data further illustrate the fact that CTL responses, as assessed by tetramer staining, are also strongly predictive of both strong CD4 T cell and humoral immune responses.

### Example 13

### Assessing the T cell Memory Response to Vaccination with LANAC

A series of experiments were performed to assess the T cell memory response to vaccination with LANAC. CD8⁺ T cells were obtained by enzymatic digestion from lung tissues of mice and analyzed using kb-ova8 tetramers after IP immunization twice with ova-LANAC. Tetramer positive cells were quantitated in 3 mice per group in control and vaccinated mice at day 5 post-immunization (FIG. 13, top two panels) and day 30 post-immunization (FIG. 13, bottom two panels). The mean percent tet⁺ cells as a % of total pulmonary CD8 T cells is plotted. Though there was a massive CTL response at 2 weeks of immunization, it could be argued that these T cells were actually short-lived and would rapidly disappear.

The CTL memory cells were examined, using tetramers, including lymphoid organs and also peripheral tissues. It is known for example, that viral infection in mice leads initially to a large expansion of Ag-specific T cells in lymphoid organs, followed by the dispersal of these long-lived memory CTL to non-lymphoid tissues, including the lungs. Exactly the same phenomenon was found to occur after immunization with ova-LANAC. In lung tissues on day 5 after the second IP immunization, a massive expansion of antigen-specific CTL was features, integers, components, or steps, but they do not preclude the presence or addition of one or more other features, integers, components, steps, or groups thereof.

1. A method comprising:
administering a composition comprising at least one ligand for a pattern recognition receptor molecule and a delivery vehicle to a subject.

2. The method of claim 1, wherein a ligand for a pattern recognition receptor comprises a ligand for a signaling pattern recognition receptor.

3. The method of claim 2, wherein said signaling pattern recognition receptor comprises at least one receptor selected from the group consisting of Toll-like receptors TLR-1, TLR-2, TLR-3, TLR-4, TLR-5, TLR-6, TLR-7, TLR-8, TLR-9, TLR-10, TLR-11 and TLR-12 and mannan-binding lectins, and macrophage mannose receptor and scavenger receptors.

4. The method of claim 3, wherein said ligand comprises a ligand for TLR-2, TLR-3 and/or TLR-9.

5. The method of claim 1, wherein a ligand for a pattern recognition receptor comprises a ligand for an endocytic pattern recognition receptor or scavenger receptor or mannose-binding receptor.

6. The method of claim 1, further comprising modulating an immune response in said subject.

7. The method of claim 6, wherein modulating an immune response comprises augmenting an immune response.

8. The method of claim 6, wherein modulating an immune response comprises down regulating an immune response.

9. The method of claim 6, wherein modulating an immune response comprises augmenting an immune response in a subject disposed of cancer.

10. The method of claim 9, wherein cancer comprises one or more selected from the group consisting of lung cancer, skin cancer, liver cancer, bone marrow cancer, leukemia, ovarian cancer, breast cancer, prostate cancer, colon cancer, lymphoma, brain cancer, renal cell cancer, and cancers of mesenchymal tissues

11. The method of claim 6, wherein modulating an immune response comprises augmenting an immune response in a subject disposed of an infectious disease.

12. The method of claim 11, wherein said infectious disease is caused by one or more organisms selected from the group consisting of a viral pathogen, a fungal pathogen, a bacterial pathogen, a rickettsial pathogen, a parasitic pathogen and a prion pathogen.

13. The method of claim 6, wherein modulating an immune response comprises augmenting or suppressing an immune response in a subject disposed of an allergic disease.

14. The method of claim 13, wherein said allergic disease is caused by an abnormal immune response against an endogenous non-self antigen.

15. The method of claim 14, wherein said non-self antigens comprise at least one of the group consisting of inhaled allergens, cutaneous allergens and oral allergens.

16. The method of claim 6, wherein modulating an immune response comprises modulating an immune response in a subject disposed of an autoimmune disease.

17. The method of claim 16, wherein said autoimmune disease is caused by an abnormal immune response against self antigens.

18. The method of claim 17, wherein the abnormal immune response against self antigens is caused by at least one antigen from the group consisting of antigens derived from the nervous system, antigens derived from the joints, antigens derived from the blood elements, antigens derived from the kidneys, and antigens derived from the eyes.

19. The method of claim 6, wherein modulating an immune response comprises modulating an immune response in a subject disposed of a disease due to abnormal production of proteins in the body.

20. The method of claim 16, wherein said autoimmune disease is caused by an abnormal production of proteins.

21. The method of claim 20, wherein the abnormal production of proteins comprises proteins selected from the group consisting of abnormal proteins in the brain, abnormal proteins of the kidneys, and abnormal proteins of the joints.

22. The method of claim 21, wherein the abnormal proteins of the brain includes abnormal protein of the brain or blood as in the case of in Alzheimer's disease.

23. The method of claim 1, wherein said administration comprises administration by at least one route selected from the group consisting of intravenously, intraperitoneally, by inhalation, subcutaneously, intradermally, intranodally, intramuscularly, intranasally, orally, rectally, intravaginally, intravesicularly, intraocularly, and topically.

24. A method comprising:
administering an agent capable of inducing an immune response against a specific cell type wherein said agent is capable of inducing an immune response against that cell type and inhibiting the normal or abnormal function of that cell type.

25. The method of claim 24, wherein said specific cell type comprises an endothelial cell for the purpose of inhibition of angiogenesis.

26. A method comprising:
administering at least one pattern recognition receptor ligand
and a delivery vehicle capable of stimulating angiogenesis and/or fibrogenesis and/or osteogenesis.

27. The method of claim 26, wherein the pattern recognition receptor ligand is complexed to the delivery vehicle.

28. The method of claim 26, wherein the pattern recognition receptor is selected from the group consisting of TLR ligands, and other pattern recognition receptors.

29. The method of claim 26, further comprising treating a subject with a wound, a bone defect or a fracture.

30. The method of claim 29, wherein the wound comprises a wound or a defect in skin or soft tissues.

31. A composition comprising:
a ligand for the pattern recognition molecule family of receptors; and
a delivery vehicle wherein said composition is capable of inducing an immune response in a subject.

32. The composition of claim 31, wherein inducing an immune response comprises inducing an innate immune response.

33. The composition of claim 32, wherein the innate immune response comprises an innate immune response by macrophages, neutrophils, NK cells, and/or dendritic cells.

34. The composition of claim 31, wherein the delivery vehicle comprises a liposome.

35. The composition of claim 34, wherein the ratio of liposome to ligand comprises about 1:1 to about 100:1 mmol liposome to mg ligand.

36. The composition of claim 34, wherein said ratio of liposomes to ligand is about 16:1 or about 8:1 mmol liposome to mg ligand.

37. The composition of claim 34, wherein said liposome comprises at least one liposome selected from the group consisting of a positively charged liposome; a negatively charged liposome; and a neutral liposome.

38. The composition of claim 31, wherein said delivery vehicle comprises any combination of liposomes.

39. The composition of claim 37, wherein said positively charged liposome is complexed to a ligand for the pattern recognition molecule family of receptors.

40. The composition of claim 34, wherein said liposome consists of a mixture of charged and neutral lipids of DOTIM (1-(2-(oleoyloxy)ethyl)-2-oleyl-3-(2-hydroxyethyl)imidazolinium) and cholesterol in a 1:1 molar ratio.

41. The composition of claim 31, wherein the delivery vehicle is non-liposomal.

42. The composition of claim 30, wherein the non-liposomal delivery vehicle comprises at least one vehicle selected from the group consisting of polypeptides, polyamines, chitosan, PEI, polyglutamic acid, protamine sulfate, and microspheres.

43. The composition of claim 34, wherein said ligand comprises a TLR ligand.

44. The composition of claim 43, wherein the TLR ligand comprises any portion of a bacterium.

45. The composition of claim 44, wherein any portion of a bacterium further comprises any portion of a bacterium that associates with a TLR.

46. The composition of claim 45, wherein said TLR ligand comprises any portion of a a bacterium that associates with at least one of the following consisting of TLR-1, TLR-2, TLR-3, TLR-4, TLR-5, TLR-6, TLR-7, TLR-8, TLR-9, TLR-10, TLR-11 and TLR-12.

47. The composition of claim 43, wherein the TLR ligand comprises any portion of a fungal organism.

48. The composition of claim 47, wherein said TLR ligand comprises any portion a fungal organism that associates with a TLR.

49. The composition of claim 37, wherein said any portion of a fungal organism further comprises any portion of yeast that associates with at least one receptor selected from the group consisting of TLR-1, TLR-2, TLR-3, TLR-4, TLR-5, TLR-6, TLR-7,TLR-8, TLR-9, TLR-10, TLR-11 and TLR-12.

50. The composition of claim 43, wherein the TLR ligand comprises any portion of a multicellular organism.

51. The composition of claim 43, wherein the TLR ligand comprises any portion of a unicellular organism.

52. The composition of claim 31, wherein said ligand comprises at least one of the following consisting of a glycoprotein, lipoprotein, glycolipid, carbohydrate, lipid, nucleic acid and/or protein or peptide sequence derived from any portion of a bacterial pathogen.

53. The composition of claim 52, further comprising any portion of a bacterial pathogen that binds at least one receptor selected from the group consisting of TLR-1, TLR-2, TLR-3, TLR-4, TLR-5, TLR-6, TLR-7, TLR-8, TLR-9, TLR-10, TLR-11 and TLR-12.

54. The composition of claim 31, wherein said ligand comprises at least one ligand selected from the group consisting of a glycoprotein, lipoprotein, glycolipid, carbohydrate, lipid, nucleic acid and and/or protein or peptide sequence derived from any portion of said fungal organism that associates with one or more selected from the group consisting of TLR-1, TLR-2, TLR-3, TLR-4, TLR-5, TLR-6, TLR-7, TLR-8, TLR-9, TLR-10, TLR-11 and TLR-12.

55. The composition of claim 31, wherein said ligand comprises a glycoprotein, lipoprotein, glycolipid, carbohydrate, lipid, nucleic acid and/or protein or peptide sequence derived from any portion of a fungal organism.

56. The composition of claim 31, wherein the ligand comprises a glycoprotein, lipoprotein, glycolipid, carbohydrate, lipid, nucleic acid and and/or protein or peptide sequence derived from any portion of a viral organism.

57. The composition of claim 31, wherein the ligand comprises a glycoprotein, lipoprotein, glycolipid, carbohydrate, lipid, nucleic acid and and/or protein or peptide sequence derived from any portion of a rickettsial organism.

58. The composition of claim 31, wherein the ligand comprises a glycoprotein, lipoprotein, glycolipid, carbohydrate, lipid, nucleic acid and and/or protein or peptide sequence derived from any portion of a parasitic organism.

59. The composition of claim 31, wherein the ligand comprises a glycoprotein, lipoprotein, glycolipid, carbohydrate, lipid, nucleic acid and and/or protein or peptide sequence derived from any portion of an arthropod organism.

60. The composition of claim 31, wherein said ligand comprises a nucleic acid encoding a TLR ligand.

61. The composition of claim 60, wherein said nucleic acid comprises at least one molecule selected from the group consisting of bacterial DNA, eukaryotic DNA, dsDNA, ssDNA a synthetic oligonucleotide, RNA, and synthetic RNA.

62. The composition of claim 61, wherein said oligonucleotide comprises at least one of poly I:C or related poly I:C oligonucleotides.

63. The composition of claim 31, wherein said ligand is a mixture of two or more different TLR ligands in ratios sufficient for eliciting an immune response.

64. The composition of claim 31, wherein said ligand consists of any molecule that associates with and/or stimulates a pattern recognition receptor.

65. The composition of claim 31, wherein said ligand comprises a synthetically generated ligand that binds to and stimulates a pattern recognition receptor.

66. The composition of claim 31, further comprising any molecule with a steroid backbone.

67. The composition of claim 60, further comprising a DNA condensing agent.

68. The composition of claim 67, wherein the DNA condensing agent is polyethylenimine (PEI).

69. A composition comprising:
at least one antigen; and
an adjuvant composition comprising a delivery vehicle; and at least one ligand for a pattern recognition receptor molecule.

70. The composition of claim 69, further comprising said antigen and said ligand for a pattern recognition molecule receptor are complexed to said delivery vehicle.

71. The composition of claim 69, wherein the ligand for the pattern recognition molecule receptor comprises a TLR receptor ligand.

72. The composition of claim 69, wherein said antigen comprises an intact microorganism.

73. The composition of claim 72, wherein said microorganism comprises at least one organism selected from the group consisting of viral organism, bacterial organism, fungal organism, protozoan organism, parasitic pathogenic organisms, rickettsial organisms, and arthropod organisms.

74. The composition of claim 69, wherein said antigen comprises at least one molecule selected from the group consisting of a protein, a peptide, a carbohydrate, a lipoprotein; a glycopeptide, a glycoprotein, a glycolipid and a lipid.

75. The composition of claim 69, wherein said antigen is a cell.

76. The composition of claim 75, wherein said cell consist of one or more of an autologous, or an allogeneic tumor cell.

77. The composition of claim 69, wherein said delivery vehicle comprises a liposome.

78. The composition of claim 69, wherein said delivery vehicle comprises lipids selected from the group consisting of multilamellar vesicle lipids, extruded liposomes and unilamellar liposomes.

79. The composition of claim 77, wherein said liposome comprises at least one of the group consisting of a positively charged liposome, a modified multilamellar liposome, a cationic liposome, a neutral liposome and a negatively charged liposome.

80. The composition of claim 69, wherein said delivery vehicle comprises at least one pair of lipids selected from the group consisting of DOTMA and cholesterol; DOTAP and cholesterol; DOTIM and cholesterol; DDAB and cholesterol.

81. The composition of claim 69, wherein said delivery vehicle comprises a non-liposomal delivery vehicle.

82. The composition of claim 81, wherein the delivery vehicle comprises at least one vehicle selected from the group consisting of polypeptides, polyamines, chitosan, PEI, polyglutamic acid, protamine sulfate and triclosan.

83. A method for vaccinating comprising:
administering to a subject a composition of an antigen; and an adjuvant composition including a delivery vehicle; and
a TLR ligand to a subject.

84. The method of claim 83, further comprising said antigen and said TLR ligand complexed to said delivery vehicle.

85. The method of claim 83, further comprising administering said composition by a route selected from the group consisting of: intravenously, intraperitoneally, by inhalation, subcutaneously, intradermally, intranodally, intramuscularly, intranasally, orally, rectally, intravaginally, intravesicularly, intraocularly, and topically.

86. The method of claim 83, further comprising augmenting an immune response in a subject disposed of cancer.

87. The method of claim 86, wherein the cancer comprises at least one cancer selected from of the group consisting of lung cancer, skin cancer, liver cancer, bone marrow cancer, ovarian cancer, breast cancer, prostate cancer, colon cancer, lymphoma, brain cancer, renal cell cancer, and cancers of mesenchymal tissues.

88. The method of claim 83, further comprising augmenting an immune response in a subject disposed of infectious disease.

89. The method of claim 88, wherein said infectious disease comprises at least one disease selected from the group consisting of a disease due to a viral pathogen; a fungal pathogen, a bacterial pathogen, a ricketssial pathogen, a parasitic pathogen, an arthrorpod pathogen, and a prion pathogen.

90. A composition comprising:
an adjuvant composition comprising
at least one antigen,
a delivery vehicle; and
at least one ligand for a pattern recognition receptor molecule.

91. The composition of claim 90, further comprising said antigen incorporated into said delivery vehicle and then mixed with a ligand for a pattern recognition molecule receptor.

92. The composition of claim 91, wherein said ligand for a pattern recognition molecule receptor comprises a TLR ligand.

93. The composition of claim 90, wherein the delivery vehicle comprises a liposome.

94. The composition of claim 93, wherein the ratio of liposome to TLR ligand is from about 1:1 to about 100:1 nmol liposome per ng TLR ligand.

95. The composition of claim 93, wherein said liposome consists of at least one molecule selected from the group consisting of a positively charged liposome, a negatively charged liposome; a cationic liposome; and a modified multilamellar liposome.

96. The composition of claim 95, wherein said cationic liposome further comprises said cationic liposomes complexed to bacterial DNA.

97. The composition of claim 90, wherein said delivery vehicle consists of a mixture of charged and neutral lipids.

98. The composition of claim 90, wherein said TLR ligand comprises any portion of a bacterium that associates with a TLR.

99. The composition of claim 90, wherein said TLR ligand comprises a bacterial cell wall component

100. The composition of claim 90, wherein said TLR ligand binds at least one receptor selected from the group consisting of TLR-1, TLR-2, TLR-3, TLR-4, TLR-5, TLR-6, TLR-7, TLR-8, TLR-9, TLR-10, TLR-11 and TLR-12.

101. The composition of claim 90, wherein said TLR ligand binds TLR 2, TLR 5 or TLR 9.

102. The composition of claim 90, wherein said TLR ligand comprises a flagellin protein.

103. The composition of claim 102, wherein said flagellin protein comprises the minimal portion of flagellin capable of binding and activating a TLR 5.

104. The composition of claim 90, wherein said TLR ligand comprises a nucleic acid.

105. The composition of claim 104, wherein said nucleic acid comprises at least one molecule selected from the group consisting of bacterial DNA, eukaryotic DNA, synthetic oligonucleotide, and RNA.

106. The composition of claim 105, wherein said RNA comprises at least one molecule selected from the group of double-stranded RNA, single-stranded RNA and synthetic RNA.

107. The composition of claim 90, wherein said TLR ligand comprises at least one of poly I:C and poly I:C related oligonucleotides that is capable of binding TLR3.

108. The composition of claim 90, wherein said TLR ligand comprises any portion of a fungal or a yeast organism.

109. The composition of claim 108, wherein the portion of a fungal or a yeast organism comprises any portion of a cell wall of the organism.

110. The composition of claim 90, wherein said TLR ligand comprises a mixture of two or more different TLR ligands in ratios sufficient for eliciting immune responses.

111. The composition of claim 90, wherein said TLR ligand consists of any ligand that associates with and/or stimulates a TLR.

112. A method of treating a subject with cancer comprising:
administering at least one ligand for a pattern recognition receptor and a delivery vehicle; in conjunction with at least one cancer therapy wherein said method elicits a response in a subject disposed of cancer.

113. The method of claim 112, wherein said cancer therapy comprises at least one therapy consisting of hyperthermia therapy, radiation therapy, chemotherapy, photodynamic therapy (PDT), surgery, ultrasound, and focused ultrasound.

114. The method of claim 112, wherein the order of administering the therapy generates different responses.

115. The method of claim 114, wherein radiation therapy is introduced first.

116. The method of claim 114, wherein radiation therapy is introduced last.

117. The method of claim 114, wherein radiation therapy is introduced concurrently.

118. The method of claim 112, wherein the pattern recognition receptor ligand comprises a nucleic acid molecule.

119. The method of claim 112, wherein the pattern recognition receptor ligand comprises bacterial DNA.

120. The method of claim 112, wherein the delivery vehicle comprises a liposome.

121. The method of claim 112, wherein the delivery vehicle comprises a non-liposomal delivery vehicle.

122. A method comprising:
coating a medical device with a composition comprising at least one ligand for a pattern recognition molecule receptor; and
a delivery vehicle.

123. The method of claim 122, wherein the medical device comprises an implanted device.

124. The method of claim 123 wherein the implanted device consists of at least one of the following devices consisting of a catheter, a stent, a mesh repair material, a Dacron vascular prothesis, a orthopedic metallic plate, a rod and a screws.

125. The method of claim 122, wherein the delivery device comprises a sustained release particle and a delivery vehicle.

126. The method of claim 125, wherein the delivery vehicle comprises a liposome.

127. The method of claim 126, wherein the liposome further comprises a liposome combined with an inert matrix or sustained release biomaterial.

128. The method of claim 127, wherein the inert matrix comprises at least one material selected from the group consisting of collagen, gelatin, PLA (define) microspheres, serum clots, and organic gels.

129. A method comprising:
administering a composition comprising at least one ligand for a pattern recognition molecule receptor;
a delivery device; and
radiation therapy to a subject.

130. The method of claim 129, wherein a ligand for a pattern recognition molecule receptor comprises a ligand for a signaling pattern recognition receptor.

131. The method of claim 129, wherein a ligand for a pattern recognition molecule receptor comprises a ligand for an pattern recognition receptor.

132. The method of claim 129, further comprising augmenting an immune response in said subject.

133. The method of claim 132, wherein augmenting an immune response comprises augmenting an immune response in a subject disposed of cancer.

134. The method of claim 133, wherein cancer comprises at least one cancer selected from the group consisting of lung cancer, skin cancer, liver cancer, bone marrow cancer, brain cancer, renal cell cancer, ovarian cancer, breast cancer, prostate cancer, cancers of mesenchymal tissues, lymphoma and colon cancer.

135. The composition of claim 129, wherein the order of administering the therapy generates different responses.

136. The composition of claim 135, wherein radiation therapy is introduced first.

137. The composition of claim 135, wherein radiation therapy is introduced last.

138. The composition of claim 135, wherein radiation therapy is introduced concurrently.

139. The method of claim 129, wherein the ligand comprises a synthetic compound capable of binding a pattern recognition receptor.

140. The method of claim 139, wherein the synthetic compound comprises immadazoquinoline.

141. A kit comprising:
a delivery container;
a delivery device;
at least one ligand for a pattern recognition receptor; and
plus or minus an antigen; wherein said ligand is capable of eliciting an immune response in a subject.

142. The kit of claim 141, further comprising one or more chemotherapy agents.

143. A method comprising:
administering a composition comprising at least one ligand for a pattern recognition molecule receptor; and
a delivery vehicle to a subject wherein said composition increases bone healing.

144. The method of claim 143, wherein the composition is administered prior to a bone graft.

145. The method of claim 143, wherein the ligand is encapsulated by an extended release material.

146. A method comprising:
administering a composition comprising at least one ligand for a pattern recognition molecule receptor; and
a delivery vehicle to a subject wherein said composition is capable of relieving injury.

147. The method of claim 146, wherein the injury comprises at least one of oxidative stress injury and/or apoptotic mediated injury.

148. The method of claim 147, wherein the injury comprises mucositis, serositis, parenchymal injury, reperfusion injury, or radio and/or chemotherapy associated injury.

149. The method of claim 146, wherein the composition further initiates an innate immune response.

150. The method of claim 146, wherein the composition is administered to a subject of advanced age.
